(19) United States
(12) Patent Application Publication
(10) Pub. No.: US 2026/0207849 A1
(43) Pub. Date: Jul. 23, 2026

(54) AUTOMATIC LOCKING AND UNLOCKING VACUUM SYRINGES, AND ASSOCIATED SYSTEMS AND METHODS

(71) Applicant: Stryker Corporation, Portage, MI (US)

(72) Inventors: Christopher Andrew Zikry, Northridge, CA (US); Benjamin Edward Merritt, San Clemente, CA (US); Parker Ozenne, Aliso Viejo, CA (US); Steven McConnell, Anaheim, CA (US); Mikayla Ann Barkley, Irvine, CA (US)

(21) Appl. No.: 19/462,580

(22) Filed: Jan. 28, 2026

Related U.S. Application Data
(63) Continuation of application No. 18/949,343, filed on Nov. 15, 2024, now Pat. No. 12,558,486.
(60) Provisional application No. 63/599,648, filed on Nov. 16, 2023.

(51) Int. Cl. A61M 5/315; A61M 5/20

(52) U.S. Cl. CPC A61M 5/31501; A61M 5/2033

(57) ABSTRACT

Disclosed herein are automatic-locking and automatic-unlocking syringes, and associated systems and methods. In some embodiments, a syringe can include a barrel, a plunger assembly slidably positioned within the barrel, and a locking assembly coupled to the barrel. The syringe is configured to (i) automatically lock the plunger assembly to the locking assembly when the plunger assembly is withdrawn through the barrel with a vacuum in the barrel, (ii) automatically unlock the plunger assembly from the locking assembly when the barrel no longer experiences vacuum and/or experiences negligible vacuum, and/or (iii) inhibit or even prevent automatic locking of the plunger assembly to the locking assembly when the plunger assembly is withdrawn through the barrel with no and/or negligible vacuum within the barrel.

### CROSS-REFERENCE TO RELATED APPLICATION

This is a continuation of U.S. patent application Ser. No. 18/949,343, filed Nov. 15, 2024, which claims the benefit of U.S. Provisional Ser. No. 63/599,648 , filed Nov. 16, 2023, each of which is incorporated herein by reference in its entirety.

### TECHNICAL FIELD

The present technology generally relates to automatically locking and unlocking syringes, such as for use in systems for treating occlusive (e.g., clot) material within a human patient, and associated systems and methods.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by occlusive material, such as clot material, tissue ischemia develops. The ischemia will progress to tissue infarction if the occlusion persists. However, infarction does not develop or is greatly limited if the flow of blood is reestablished rapidly. Failure to reestablish blood flow can accordingly lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT commonly occurs where there is a propensity for stagnated blood (e.g., long-distance air travel, immobility, etc.) and clotting (e.g., cancer; recent surgery, such as orthopedic surgery, etc.). DVT can obstruct drainage of venous blood from the legs, leading to swelling, ulcers, pain, and infection. DVT can also create a reservoir in which blood clots can collect and then travel to other parts of the body, including the heart, lungs, brain (which may cause a stroke), abdominal organs, and/or extremities.

In the pulmonary circulation, occlusive material can cause harm by obstructing pulmonary arteries—a condition known as pulmonary embolism (PE). If the obstruction is upstream, in the main or large branch pulmonary arteries, it can severely compromise total blood flow within the lungs, and therefore the entire body, and result in low blood pressure and shock. If the obstruction is downstream, in large to medium pulmonary artery branches, it can prevent a significant portion of the lung from participating in the exchange of gases to the blood resulting in low blood oxygen and buildup of blood carbon dioxide.

Various systems exist for performing a thrombectomy or removing occlusive material to reestablish blood flow within a patient. Some of these systems use (i) a syringe or other pressure source to generate and store a vacuum and (ii) a fluid control device, such as a stopcock, to apply the vacuum to a catheter to aspirate the occlusive material. To maintain the vacuum pressure generated in the syringe before opening the fluid control device, a plunger of the syringe may be selectively or automatically locked in a withdrawn position. However, such syringes require a user input to lock and/or unlock the plunger.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a partially schematic side view of a clot treatment system in accordance with embodiments of the present technology.

FIGS. 2A and 2B are an isometric view and an exploded view, respectively, of a syringe of the clot treatment system of FIG. 1 in accordance with embodiments of the present technology.

FIG. 3 is a partially-transparent side view of a plunger assembly of the syringe of FIGS. 2A and 2B in accordance with embodiments of the present technology.

FIGS. 4A and 5A are cross-sectional side views of the syringe of FIGS. 2A and 2B in a first position and a second position, respectively, in accordance with embodiments of the present technology.

FIGS. 4B and 5B are enlarged side views of a distal portion of the plunger assembly of the syringe of FIGS. 2A and 2B in the first position and the second position, respectively, in accordance with embodiments of the present technology.

FIG. 6 is a side view of the distal portion of the plunger assembly shown in FIGS. 4B and 5B in an intermediate position between the first position and the second position in accordance with embodiments of the present technology.

FIG. 7 is an isometric view of a locking assembly of the syringe of FIGS. 2A and 2B in accordance with embodiments of the present technology.

FIGS. 8A and 8B are enlarged cross-sectional side views of the syringe of FIGS. 2A and 2B with the plunger assembly in a withdrawn position and locked to the locking assembly and unlocked from the locking assembly, respectively, in accordance with embodiments of the present technology.

FIG. 9 is a table including various views of the syringe of FIGS. 2A and 2B at different vacuum levels in accordance with embodiments of the present technology.

FIG. 10 is an isometric view of a syringe in accordance with additional embodiments of the present technology.

FIGS. 11A and 11B are side views of a syringe that can be used in the clot treatment system of FIG. 1 in a first position and a second position, respectively, in accordance with additional embodiments of the present technology.

FIGS. 12A and 13A are partially-transparent side views of a plunger assembly of the syringe of FIGS. 11A and 11B in a first position and a second position, respectively, in accordance with embodiments of the present technology.

FIGS. 12B and 13B are enlarged side views of a distal portion of a plunger assembly of the syringe of FIGS. 12A and 13A in the first position and the second position, respectively, in accordance with embodiments of the present technology.

FIGS. 14A and 14B are cross-sectional side views of the syringe of FIGS. 11A and 11B in an unlocked position and a locked position, respectively, in accordance with additional embodiments of the present technology.

FIG. 15 is a cross-sectional view of a locking assembly of the syringe of FIGS. 11A and 11B in accordance with embodiments of the present technology.

### DETAILED DESCRIPTION

The present technology is generally directed to syringes for use in clot treatment systems, and associated systems and methods. In some embodiments, a syringe in accordance with the present technology can include a barrel, a plunger assembly slidably positioned within the barrel, and a locking assembly coupled to the barrel. The syringe is configured to (i) automatically lock the plunger assembly to the locking assembly when the plunger assembly is withdrawn through the barrel with a vacuum in the barrel, (ii) automatically unlock the plunger assembly from the locking assembly when the barrel no longer experiences vacuum and/or experiences negligible vacuum, and/or (iii) inhibit or even prevent automatic locking of the plunger assembly to the locking assembly when the plunger assembly is withdrawn through the barrel with no and/or negligible vacuum within the barrel. In some aspects of the present technology, the automatic-locking and automatic-unlocking syringe can simplify a clot removal procedure using the syringe by not requiring user input to either lock or unlock the position of the plunger assembly relative to the barrel during the clot removal procedure.

In some embodiments, an automatic-locking and automatic-unlocking syringe comprises a barrel, a plunger assembly slidably positioned within the barrel, and a locking assembly coupled to the barrel. The plunger assembly can be movable between a first position and a second position. The plunger assembly can be configured to be withdrawn through the barrel to generate vacuum pressure in the barrel. The plunger assembly can be configured to move from the first position to the second position in response to the vacuum pressure, and to move from the second position to the first position in response to the vacuum pressure being released. The plunger assembly can be configured to lock to the locking assembly in the second position such that the locking assembly inhibits movement of the plunger assembly through the barrel, and to unlock from the locking assembly in the first position such that the locking assembly permits movement of the plunger assembly through the barrel.

In some embodiments, an automatic-locking and automatic-unlocking syringe comprises a barrel and a plunger assembly slidably positioned within the barrel. The plunger assembly can comprise (i) a handle assembly including a first shaft having a distal portion, (ii) a seal assembly including a second shaft and a seal head coupled to the second shaft and having a sealing member positioned to sealingly engage the barrel, and (iii) a biasing member operably coupling the first shaft to the second shaft. The handle assembly can be configured to be withdrawn relative to the barrel to withdraw the seal assembly and the sealing member through the barrel to generate vacuum pressure within the barrel. The vacuum pressure can generate a vacuum force on the seal assembly greater than a biasing force of the biasing member such that the biasing member compresses and the seal head moves away from the distal portion of the first shaft.

In some embodiments, an automatic-locking and automatic-unlocking syringe comprises a barrel and a plunger assembly slidably positioned within the barrel. The plunger assembly can include a handle assembly and a seal assembly. The handle assembly can include a first shaft having a distal portion and a locking recess formed at the distal portion. The seal assembly can include a shield member. The seal assembly can be movable relative to the handle assembly between (i) a first position in which the shield member radially shields the locking recess and (ii) a second position in which the shield member is axially spaced apart from the locking recess.

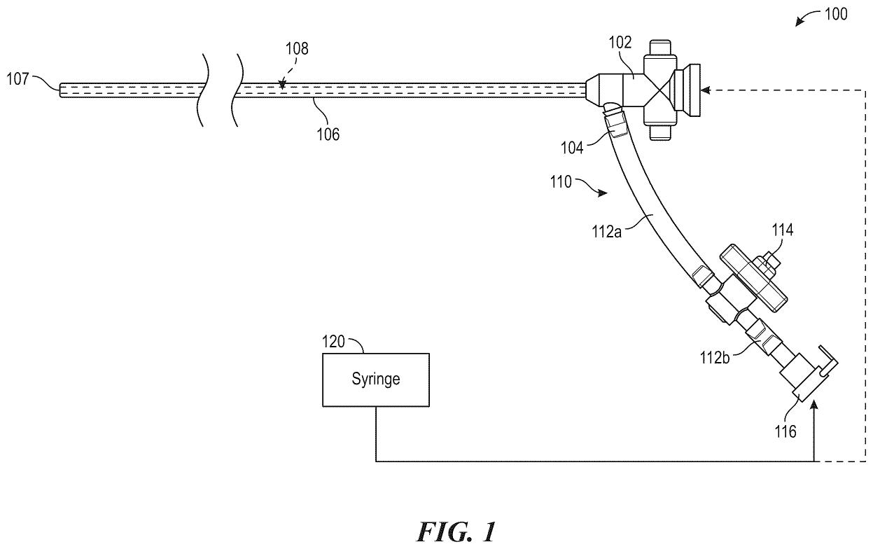

Certain details are set forth in the following description and in FIGS. 1-15 to provide a thorough understanding of various embodiments of the present technology. In other instances, well-known structures, materials, operations, and/or systems often associated with intravascular procedures, clot removal procedures, clot treatment systems, clot treatment devices, fluid control devices, syringes, vacuum-locking syringes, blood-filtering syringes, catheters, and/or the like are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. Moreover, although many of the devices and systems are described herein in the context of removing and/or treating clot material, the present technology can be used to remove and/or treat other unwanted material in addition or alternatively to clot material, such as thrombi, emboli, plaque, intimal hyperplasia, post-thrombotic scar tissue, etc. Accordingly, the terms “clot” and “clot material” as used herein can refer to any of the foregoing materials and/or the like.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

In the Figures, identical reference numbers identify identical, or at least generally similar, elements. To facilitate the discussion of any particular element, the most significant digit or digits of any reference number refers to the Figure in which that element is first introduced. For example, tubing assembly 110 is first introduced and discussed with reference to FIG. 1.

To the extent any materials incorporated herein by reference conflict with the present disclosure, the present disclosure controls.

FIG. 1 is a partially schematic side view of a clot treatment system 100 (“the system 100”) in accordance with embodiments of the present technology. The system 100 can also be referred to as an aspiration assembly, a vascular access system, a clot removal system, a thrombectomy system, and/or the like. In the illustrated embodiment, the system 100 includes a tubing assembly 110 fluidly coupled to a catheter 106 via a valve 102. In some embodiments, the catheter 106 is an elongate member (e.g., a sheath, a shaft) configured to be inserted into and through a patient's vasculature and used to, for example, remove or otherwise treat clot material therein. In other embodiments, the catheter 106 can be an introducer sheath configured to be inserted through the skin and tissue tract of the patient to provide an access site through which other components (e.g., other catheters used to treat clot material) can traverse to easily access the vasculature. Accordingly, while referred to as “catheter 106,” the catheter 106 can comprise an introducer sheath, an access sheath, and/or another type of elongate member configured to be inserted through the skin and tissue tract and/or to traverse the vasculature of a patient. The catheter 106 can be a large bore catheter, having, for example, a size equal to or greater than 16 French (Fr), such as 20 Fr, 22 Fr, 24 Fr, 26 Fr, 28 Fr, 30 Fr, 32 Fr, and/or the like. In general, the system 100 (i) can include features generally similar in structure and/or function, or identical in structure and/or function, to those of the clot treatment systems described in detail in U.S. patent application Ser. No. 16/536,185, now U.S. Pat. No. 11,559,382, filed Aug. 8, 2019, and titled “SYSTEM FOR TREATING EMBOLISM AND ASSOCIATED DEVICES AND METHODS,” which is incorporated herein by reference in its entirety, and/or (ii) can be used to treat/remove clot material from a patient (e.g., a human patient) using any of the methods described in detail therein.

The catheter 106 further defines a lumen 108 (shown in dashed line in FIG. 1) extending entirely therethrough, e.g., from the valve 102 to a distal terminus 107 of the catheter 106. The catheter 106 can have varying lengths, flexibilities, shapes, thicknesses, and/or other properties along its length. For example, the catheter 106 can comprise one or more coils, braids, and/or other structures positioned between one or more liner layers (e.g., an inner liner layer and an outer liner layer). In some embodiments, the catheter 106 can include several features generally similar or identical in structure and/or function to any of the catheters described in (i) U.S. patent application Ser. No. 17/529,018, titled “CATHETERS HAVING SHAPED DISTAL PORTIONS, AND ASSOCIATED SYSTEMS AND METHODS,” and filed Nov. 17, 2021, (ii) U.S. patent application Ser. No. 17/529,064, titled “CATHETERS HAVING STEERABLE DISTAL PORTIONS, AND ASSOCIATED SYSTEMS AND METHODS,” and filed Nov. 17, 2021, (iii) U.S. patent application Ser. No. 18/159,507, titled “ASPIRATION CATHETERS HAVING GROOVED INNER SURFACE, AND ASSOCIATED SYSTEM AND METHODS,” and filed Jan. 25, 2023, and/or (iv) U.S. patent application Ser. No. 18/463,960, titled “CATHETERS HAVING MULTIPLE COIL LAYERS, AND ASSOCIATED SYSTEMS AND METHODS,” and filed Sep. 8, 2023, each of which is incorporated by reference herein in its entirety.

The valve 102 is fluidly coupled to the lumen 108 of the catheter 106 and can be integral with or coupled to the catheter 106 such that these components move together. In some embodiments, the valve 102 is a hemostasis valve that is configured to maintain hemostasis during a clot treatment procedure by preventing fluid flow in a proximal direction through the valve 102 as various components such as dilators, delivery sheaths, pull members, guidewires, interventional devices, other aspiration catheters, and so on are inserted through the valve 102 to be delivered through the catheter 106 to a treatment site in a blood vessel. The valve 102 can include a branch or side port 104 configured to fluidly couple the lumen 108 of the catheter 106 to the tubing assembly 110. In some embodiments, the valve 102 can be a valve of the type disclosed in U.S. patent application Ser. No. 16/536,185, titled “HEMOSTASIS VALVES AND METHODS OF USE,” and filed Aug. 30, 2018, which is incorporated herein by reference in its entirety.

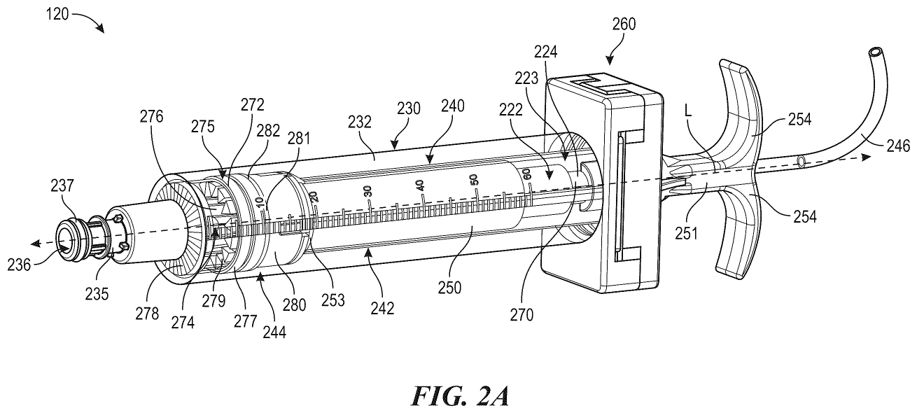

In the illustrated embodiment, the tubing assembly 110 fluidly couples the catheter 106 to a syringe 120. The syringe 120 can be configured to generate (e.g., form, create, charge, build-up) a vacuum (e.g., negative relative pressure) and store the vacuum for subsequent application to the catheter 106. Further details of the syringe 120 are described in detail below with reference to FIGS. 2A-9. The tubing assembly 110 can include one or more tubing sections 112 (individually labeled as a first tubing section 112a and a second tubing section 112b), at least one valve or fluid control device 114, and at least one connector 116 (e.g., a Toomey tip connector) for fluidly coupling the tubing assembly 110 to the syringe 120 and/or other suitable components. In some embodiments, the connector 116 is a quick-release connector (e.g., a quick disconnect fitting) that enables rapid coupling/decoupling of the catheter 106 and the fluid control device 114 to/from the syringe 120. The tubing assembly 110 and the catheter 106 can have a same or substantially same inner dimension to, for example, define a lumen or flow path of uniform or substantially uniform diameter extending from the distal terminus 107 of the catheter to the syringe 120. In some embodiments, the fluid control device 114 is fluidly coupled to (i) the side port 104 of the valve 102 via the first tubing section 112a and (ii) the connector 116 via the second tubing section 112b. The fluid control device 114 is externally operable by a user to regulate the flow of fluid therethrough and, specifically, from the lumen 108 of the catheter 106 to the syringe 120. For example, the fluid control device 114 can be transitioned between (i) a first or closed configuration in which the fluid control device 114 inhibits or even prevents fluid flow therethrough and (ii) a second or open configuration in which fluid can flow through the fluid control device 114.

During a clot removal procedure, at least a portion of the system 100, including at least a portion of the catheter 106, can be inserted through the vasculature of a patient to treat clot material therein. In some embodiments, the system 100 is inserted to a target treatment location proximate to the clot material through an introducer sheath that traverses the skin and tissue of the patient to provide an access site. The fluid control device 114 can be in the closed position during insertion of the catheter 106. After positioning the catheter 106 at the treatment location and with the fluid control device 114 in the closed position, a user/operator can generate a vacuum in the syringe 120 by, for example, fluidly coupling the syringe 120 to the connector 116 and withdrawing a plunger of the syringe 120. In this manner, a vacuum is charged within the syringe 120 (e.g., a negative pressure is maintained) before the syringe 120 is fluidly connected to the lumen 108 of the catheter 106. To aspirate the lumen 108 of the catheter 106, the user can actuate (e.g., open) the fluid control device 114 to fluidly connect the syringe 120 to the catheter 106 and thereby apply or release the vacuum stored in the syringe 120 to the lumen 108 of the catheter 106. Opening of the fluid control device 114 instantaneously or nearly instantaneously applies the stored vacuum pressure to the tubing assembly 110 and the catheter 106, thereby generating a suction pulse throughout the catheter 106 that can aspirate the clot material into the catheter 106. In some embodiments, the vacuum from the syringe 120 is applied with the fluid control device 114 in an open position (e.g., to provide continuous vacuum). That is, the user can generate the vacuum in the syringe 120 while the fluid control device 114 is open (e.g., while the syringe 120 is fluidly connected to the lumen 108 of the catheter 106) to thereby aspirate the clot material while also simultaneously generating the vacuum-that is, for example, without or substantially without storing the vacuum in the syringe 120.

FIGS. 2A and 2B are an isometric view and an exploded view, respectively, of the syringe 120 of FIG. 1 in accordance with embodiments of the present technology. The syringe 120 can be referred to as a pressure source, a vacuum syringe, an automatic-locking syringe, an automatic-unlocking syringe, a filtering syringe, an automatic-locking and automatic-unlocking syringe, an automatic-locking and automatic-unlocking vacuum syringe, a vacuum-locking syringe, a vacuum-unlocking syringe, and/or the like. In some embodiments, the syringe 120 can include some features generally similar in structure and/or function, or identical in structure and/or function, to those of any of the syringes described in U.S. patent application Ser. No. 17/396,426, titled “AUTOMATICALLY-LOCKING VACUUM SYRINGES, AND ASSOCIATED SYSTEMS AND METHODS,” and filed Aug. 8, 2021, and/or U.S. patent application Ser. No. 18/192,855, titled “BLOOD-FILTERING DEVICES FOR USE WITH CLOT TREATMENT SYSTEMS,” and filed Mar. 30, 2023, each of which is incorporated by reference herein in its entirety.

Referring to FIGS. 2A and 2B, in the illustrated embodiment the syringe 120 includes a plunger assembly 240 slidably positioned within a barrel 230. The barrel 230 is shown as partially transparent in FIG. 2A for clarity. Referring to FIG. 2B, the barrel 230 can include a barrel portion 232 (e.g., a cylindrical portion) extending between a proximal flange 231 (e.g., a proximal end portion) and a distal tip 233 (e.g., a distal end portion). In some embodiments, the barrel portion 232 can have a volume of about 60 cc or greater than 60 cc. The tip 233 is configured to be releasably or permanently coupled to an adaptor 235. Referring to FIGS. 2A and 2B, in some embodiments the adaptor 235 can define a bore 236 having a size (e.g., and corresponding inner diameter) equal to or greater than 16 Fr, 20 Fr, 22 Fr, 24 Fr, 26 Fr, 28 Fr, 30 Fr, 32 Fr, and/or the like. In the illustrated embodiment, the adaptor 235 is a Toomey-tip adaptor having a sealing member 277 (e.g., an O-ring) extending around an exterior surface thereof for sealingly engaging (e.g., connecting to) a Toomey fitting or Toomey adaptor, such as the connector 116 of FIG. 1. In other embodiments, the adaptor 235 can be omitted and the tip 233 (FIG. 2B) of the barrel 230 can be directly coupled to another device or system (not shown), and/or the adaptor 235 can be another type of adaptor such as, for example, a Luer lock, Lock slip, and/or needle.

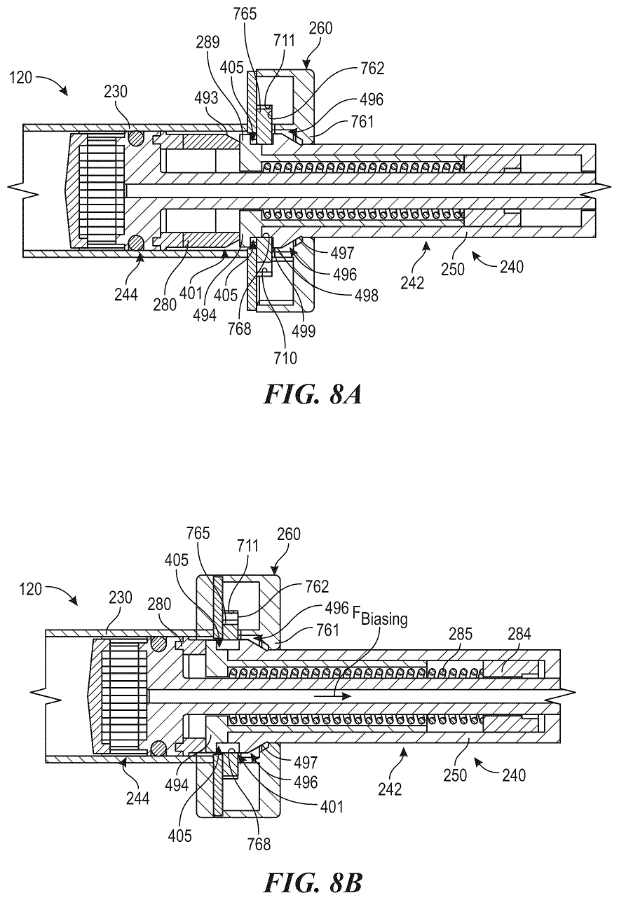

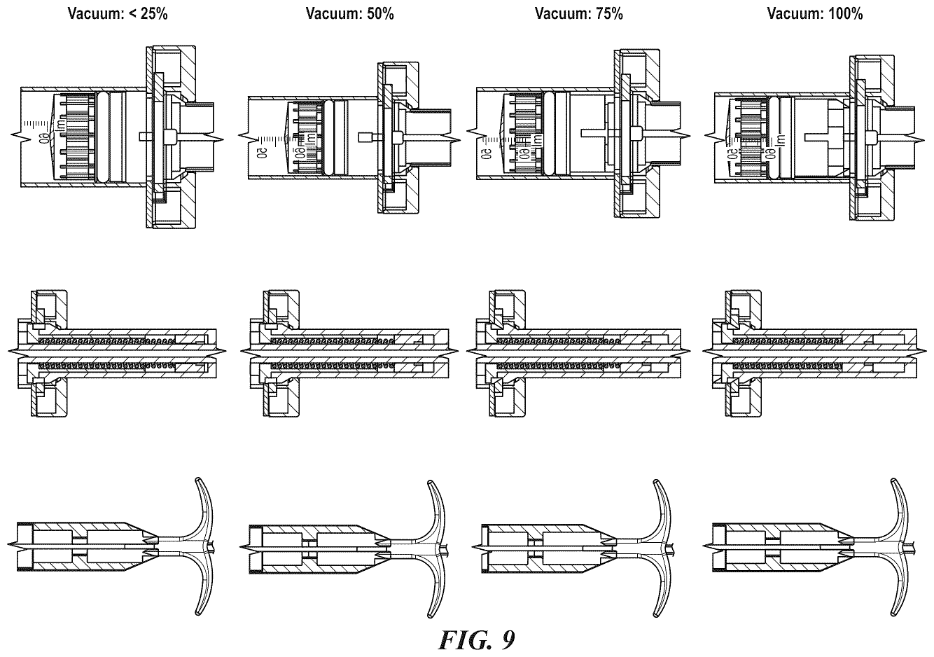

In the illustrated embodiment, the plunger assembly 240 includes a handle assembly 242 (which can also be referred to as a first assembly, a locking assembly, and/or the like) operably coupled to a seal assembly 244 (which can also be referred a second assembly, a filter assembly, a seal and filter assembly, an unlocking assembly, and/or the like). The syringe 120 further includes a locking assembly 260 coupled to the flange 231 (FIG. 2B) of the barrel 230. The locking assembly 260 is configured to selectively lock the plunger assembly 240 in a withdrawn position under vacuum pressure as described in further detail below with reference to FIGS. 8A and 9.

The handle assembly 242 includes a first shaft 250 (e.g., an elongate member, a tube, a column) extending between a proximal end portion 251 and a distal end portion 253 and defining a lumen 252 (FIG. 2B). The proximal end portion 251 can be configured (e.g., shaped, sized) to be grasped by a user for withdrawing (e.g., retracting, pulling) and/or depressing (e.g., advancing, pushing) the plunger assembly 240 through the barrel 230 during operation of the syringe 120. For example, in the illustrated embodiment the proximal end portion 251 includes a pair of opposing flanges 254 extending radially outward from a longitudinal axis L (FIG. 2A) of the syringe 120 and forming a handle configured to be grasped by a hand of the user.

The seal assembly 244 includes a second shaft 270 (partially obscured in FIG. 2A; e.g., an elongate member, a tube, a column) having a proximal end portion 271 (FIG. 2B) and a distal end portion 273 (FIG. 2B). The distal end portion 273 of the second shaft 270 can be coupled to (e.g., integrally formed, releasably or permanently attached to) a sealing head 272. The second shaft 270 defines a lumen (obscured in FIGS. 2A and 2B) extending therethrough and having a proximal opening at the proximal end portion 271 and a distal opening 274 at the sealing head 272. In the illustrated embodiment, the sealing head 272 has an annular shape including a circumferential groove 275 and a distal face 276. The distal opening 274 is formed through the distal face 276 and can, for example, be generally aligned along the longitudinal axis L (FIG. 2A). An outlet tube 246 can extend partially through the lumen 252 (FIG. 2B) of the first shaft 250 from the proximal end portion 251 of the first shaft 250 and be coupled to the proximal end portion 271 (FIG. 2B) of the second shaft 270 to fluidically couple the lumen of the second shaft 270 to the outlet tube 246 such that a fluid flow path extends through (i) the distal opening 274 in the sealing head 272, (ii) the lumen of the second shaft 270, and (iii) the outlet tube 246.

The circumferential groove 275 in the sealing head 272 can receive a sealing member 277 therein, such as an O-ring. The sealing member 277 is configured to sealingly engage an interior surface of the barrel portion 232—even as the plunger assembly 240 moves through the barrel 230—to, for example, define a sealed volume (e.g., of negative/vacuum pressure) within the barrel 230.

In the illustrated embodiment, the seal assembly 244 further includes a distal cap 278 and a filter 279 positioned between the cap 278 and the sealing head 272 (e.g., the distal face 276 of the sealing head 272). The cap 278 can have an annular shape and be impermeable to blood or other liquid flow. The filter 279 can have a circumferential shape and, in some embodiments, can include a plurality of folds or pleats circumferentially disposed thereabout. The filter 279 and the cap 278 can extend entirely about the distal face 276 of the sealing head 272 to enclose the distal opening 274. The filter 279, the cap 278, and the sealing head 272 can be coupled together (e.g., integrally formed, releasably or permanently attached together) such that these components are configured to move together through the barrel 230.

The filter 279 can be permeable to blood but impermeable to the clot material by, for example, including a plurality of pores sized to allow blood to pass therethrough but to inhibit clot material from passing therethrough. When the syringe 120 is used in a clot removal procedure on a patient, such as described in detail above with reference to FIG. 1, clot material and blood can be aspirated into the barrel 230. To filter the clot material from the blood, the plunger assembly 240 can be depressed through the barrel 230 to drive the sealing head 272 and the sealing member 277 distally through the barrel 230 to thereby increase the pressure in the barrel 230. The increased pressure can drive the blood (i) radially through the filter 279, (ii) proximally through the distal opening 274 in the sealing head 272, (iii) proximally through the lumen of the second shaft 270, and (iv) proximally through the outlet tube 246. Alternatively or additionally, a source of negative pressure (e.g., another syringe) can be coupled to the outlet tube 246 and activated to generate negative pressure that acts to draw the blood (i) radially through the filter 279, (ii) proximally through the distal opening 274 in the sealing head 272, (iii) proximally through the lumen of the second shaft 270, and (iv) proximally through the outlet tube 246. The filtered blood can be collected in a reservoir, container, aspiration source, etc., for reintroduction into the patient to minimize blood loss during the clot removal procedure. In some embodiments, such filtration components of the syringe 120 can include some features generally similar in structure and/or function, or identical in structure and/or function, to those of any of the syringes described in U.S. patent application Ser. No. 18/192,855, titled “BLOOD-FILTERING DEVICES FOR USE WITH CLOT TREATMENT SYSTEMS,” and filed Mar. 30, 2023, which is incorporated by reference herein in its entirety.

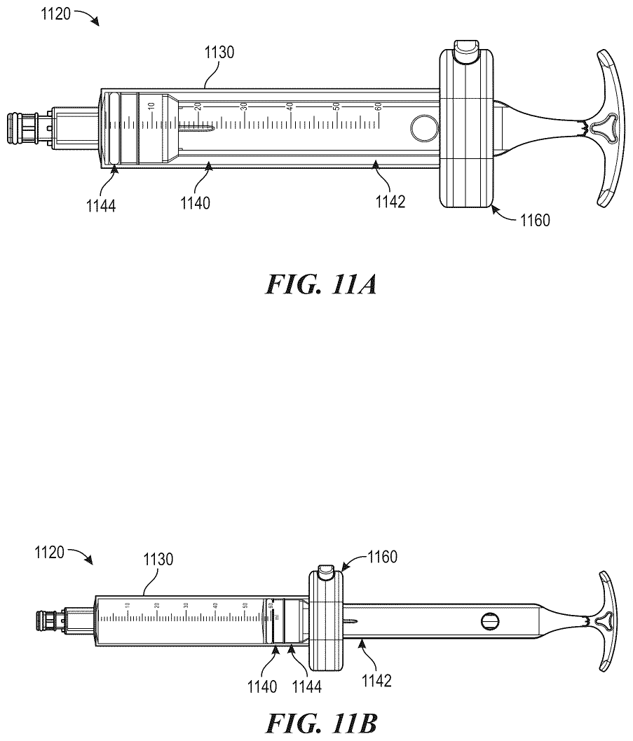

In other embodiments, the filter 279, the cap 278, the lumen in the second shaft 270, and/or the outlet tube 246 can be omitted. That is, the syringe 120 can be a non-filtering syringe. For example, FIGS. 11A-14 described in detail below illustrate a non-filtering syringe in accordance with embodiments of the present technology. In such embodiments, the seal assembly 244 can comprise only the second shaft 270 (e.g., without a lumen therethrough), the sealing head 272 (e.g., without a distal opening therethrough), and the sealing member 277.

In the illustrated embodiment, the seal assembly 244 further includes an unlocking member 280 coupled to the sealing head 272. More specifically, the unlocking member 280 (which can also be referred to as a shield member, an unlocking and shield member, and/or the like) can include a distal face or end portion 281 coupled to (e.g., attached to, fixed to, integrally formed with) a proximal face or end portion of 282 of the sealing head 272. Referring to FIG. 2B, the handle assembly 242 can further include an elongate annular insert member 283 configured to be coupled to the distal end portion 253 of the first shaft 250 and to extend at least partially through the lumen 252 of the first shaft 250. The seal assembly 244 can further include an annular mount member 284 slidably positioned within the lumen 252 of the first shaft 250 and coupled to second shaft 270. A biasing member 285, such as a compression spring, can be coupled between the insert member 283 and the mount to member 284 to operatively couple the seal assembly 244 to the handle assembly 242. As described in greater detail below, the insert member 283 constrains travel (e.g., distal movement) of the mount member 284. Accordingly, in some embodiments the insert member 283 need not extend through the lumen 252 of the first shaft 250 (e.g., where the biasing member 285 is configured to compress and bottom out to constrain travel of the mount member 284).

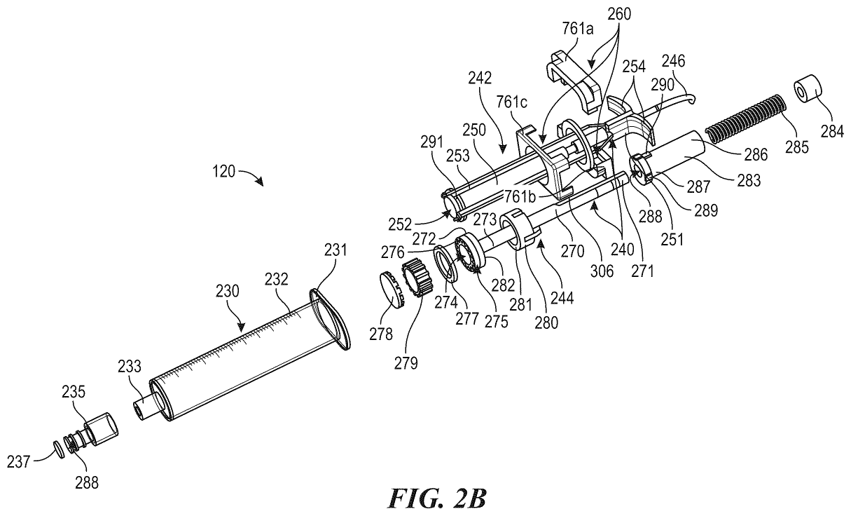

FIG. 3 is a partially-transparent side view of the plunger assembly 240 in accordance with embodiments of the present technology. The first shaft 250 of the handle assembly 242 and the unlocking member 280 of the seal assembly 244 are shown as partially transparent in FIG. 3 for clarity. Referring to FIGS. 2B and 3, the insert member 283 can have a proximal end portion 286, a distal end portion 287, a lumen 288 (FIG. 2B) extending between the proximal end portion 286 and the distal end portion 287, and a flange 289 extending circumferentially about the distal end portion 287. The flange 289 can be coupled (e.g., fixedly coupled) to the distal end portion 253 of the first shaft 250 via one or more tabs 290 that engage corresponding recesses or detents 291 at the distal end portion 253 of the first shaft 250. In some embodiments, the flange 289 includes two of the tabs 290 on opposing circumferential portions of the flange 289 (e.g., spaced apart by about 180 degrees) that engage with a corresponding pair of the detents 291 while, in other embodiments, the flange 289 includes more or fewer of the tabs 290 and/or the insert member 283 can be coupled to the first shaft 250 in different manners (e.g., via fasteners, via adhesive, integrally formed).

Referring to FIG. 3, the insert member 283 extends at least partially through the lumen 252 of the first shaft 250. The second shaft 270 of the seal assembly 244 extends from the sealing head 272 through the annular unlocking member 280, through the lumen 288 (FIG. 2B) of the insert member 283, through the annular mount member 284, and terminates at the proximal end portion 271 within the lumen 252 of the first shaft 250 proximal to the mount member 284. The mount member 284 is positioned proximal to the proximal end portion 286 of the insert member 283 within the lumen 252 of the first shaft 250 and is slidably positioned within the lumen 252. The mount member 284 can be coupled (e.g., fixedly coupled) to the second shaft 270 within the lumen 252 such that these components are configured to move together. In some embodiments, the mount member 284 includes one or more protrusions (obscured in FIG. 3) on an inner surface thereof that engage a corresponding one or more detents or tracks 306 (also shown in FIG. 2B) formed in the second shaft 270 to couple the mount member 284 to the second shaft 270. The biasing member 285 extends over (e.g., is coiled over) the second shaft 270 within the lumen 252 of the first shaft 250 and has a proximal end portion (obscured in FIG. 3) fixed to the mount member 284 and a distal end portion (obscured in FIG. 3) fixed to the insert member 283. In some embodiments, the biasing member 285 extends at least partially through the lumen 288 (FIG. 2B) of the insert member 283 and is fixed therein.

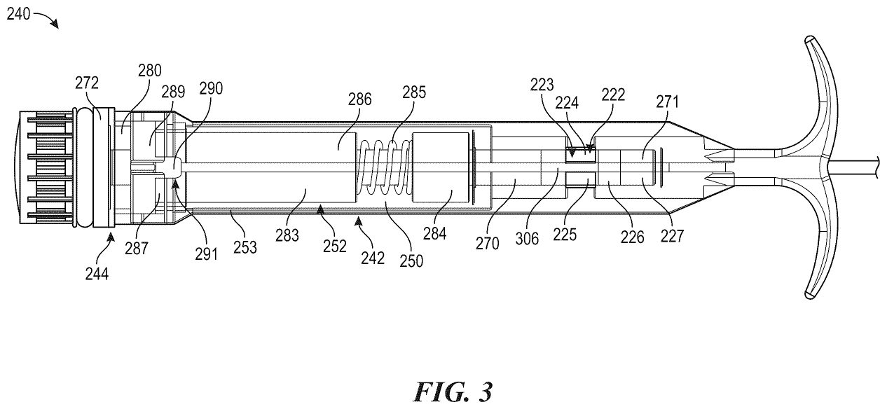

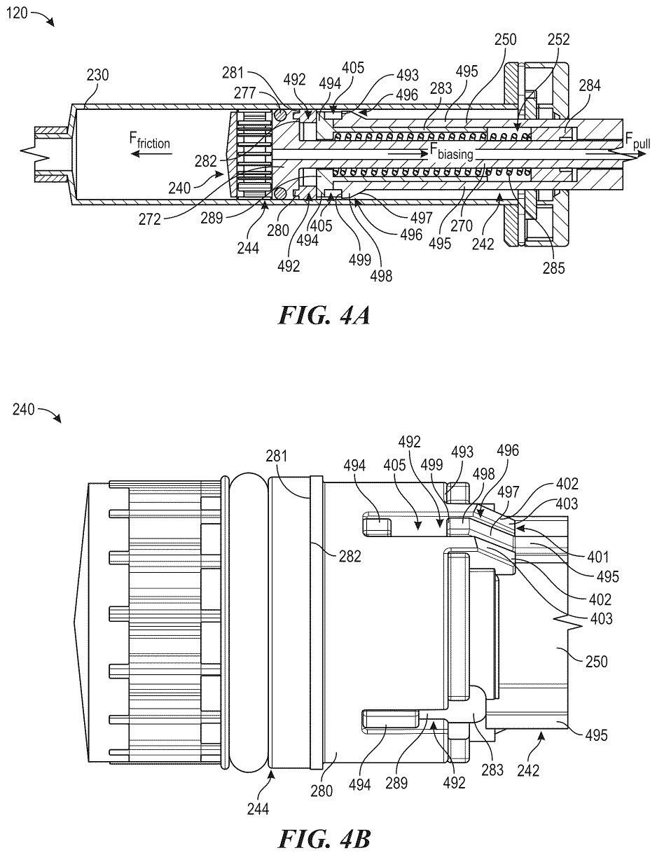

FIGS. 4A and 5A are cross-sectional side views of the syringe 120 in a first position (which can also be referred to as a shielded position, a non-vacuum position, and/or the like; as shown in FIGS. 2A and 3) and a second position (which can also be referred to as a locking position, a vacuum position, and/or the like), respectively, in accordance with embodiments of the present technology. FIGS. 4B and 5B are corresponding enlarged side views of a distal portion of the plunger assembly 240 in the first position and the second position, respectively, in accordance with embodiments of the present technology.

Referring to FIGS. 4A-5B together, the unlocking member 280 of the seal assembly 244 can include one or more grooves or channels 492 formed therein and extending distally from a proximal face or end portion 493 of the unlocking member 280 at least partially toward the distal end portion 281 thereof parallel to the longitudinal axis L (FIG. 2A). In the illustrated embodiment, the insert member 283 includes one or more projections or tabs 494 extending radially outward from the flange 289. The tabs 494 can be aligned with corresponding ones of the channels 492 and can slidingly move through the channels 492 between the first position (FIGS. 4A and 4B) and the second position (FIGS. 5A and 5B).

In the illustrated embodiment, the first shaft 250 includes one or more rails 495 extending along an exterior surface thereof and aligned with corresponding ones of the channels 492 and tabs 494. One or more of the rails 495 can include a lock feature 496 at a distal end portion thereof. In the illustrated embodiment, the lock features 496 include (i) a ramp portion 497, (ii) a plateau portion 498 extending from the ramp portion 497, and (iii) a stop surface 499 extending from the plateau portion 498. The ramp portion 497 can extend/slope at angle relative to the longitudinal axis L (FIG. 2A) in a direction (i) away from the longitudinal axis L and (ii) distally toward the unlocking member 280. The plateau portion 498 can extend away from the ramp portion 497 in a direction toward the unlocking member 280 and generally parallel to the longitudinal axis L. The stop surface 499 can extend away from the plateau portion 498 in a direction generally perpendicular to the plateau portion 498 and the longitudinal axis L. The stop surface 499 of the lock features 496 and a corresponding one of the tabs 494 can together define a locking recess 405. In some embodiments, the tabs 494 can be omitted such that the locking recesses 405 are defined distal of the stop surfaces 499 of the lock features 496. In some embodiments, the lock features 496 can compromise a tab, flat face, half disc, and/or other feature extending from the exterior surface of the first shaft 250.

The unlocking member 280 can further include one or more unlock features 401 (obscured in FIG. 4A) aligned with corresponding ones of the lock features 496. In the illustrated embodiment, the unlock features 401 include a pair of spaced apart rails or projections 402 extending from the proximal end portion 493 of the unlocking member 280 and each having a ramp portion 403 that can extend/slope at angle relative to the longitudinal axis L (FIG. 2) in a similar or identical manner to the ramp portion 497 of the lock features 496. The projections 402 extend the channel 492 of the unlocking member 280. In other embodiments, the lock features 496 and/or the unlock features 401 can have different arrangements/configurations. For example, the lock features 496 can comprise a pair of spaced apart rails or projections that extend from the unlocking member 280, and the unlock features 401 can include a ramp portion, a plateau portion, and a stop surface. In the first position shown in FIGS. 4A and 4B, the tabs 494 and the locking features 496 of the handle assembly 242 are positioned within corresponding ones of the channels 492 formed in/by the unlocking member 280 and the unlock features 401 such that the locking recesses 405 are covered (e.g., radially shielded) by the unlocking member 280. In the second position shown in FIGS. 5A and 5B, the tabs 494 and the lock features 496 of the handle assembly 242 are translated outside of the channels 492 such that the locking recesses 405 are uncovered (e.g., radially unshielded) by the unlocking member 280 and accessible.

In some embodiments, the unlocking member 280 only includes one of the channels 492 and one of the unlock features 401, and the handle assembly 242 only includes one of the rails 495, one of the lock features 496, and one of the tabs 494 (collectively a “locking and unlocking mechanism”)—each aligned with one another. In some embodiments, the plunger assembly 240 can include multiple ones of the locking and unlocking mechanisms—such as a pair of the locking and unlocking mechanisms on opposing circumferential portions of the plunger assembly 240 (e.g., spaced apart by about 180 degrees) as shown in FIGS. 4A, 5A, and 5B. In some embodiments, the plunger assembly 240 can include one or more of the channels 492, tabs 494, and rails 495 in an aligned configuration without a corresponding lock feature 496 or unlock feature 401.

Referring to FIG. 4A, the biasing member 285 can be configured (e.g., shaped, sized, formed) to allow the plunger assembly 240 to move between the first position (FIG. 4A) and the second position (FIG. 5A) based on (e.g., in response to) an amount of vacuum pressure within the barrel 230. This operation of the plunger assembly 240 is best understood in view of the various forces acting on the plunger assembly 240 as the plunger assembly 240 is withdrawn through the barrel 230. For example, retraction of the handle assembly 242 generates (i) a pull force Fₚᵤₗₗ in the proximal direction and (ii) a friction force F_{friction} in the opposite, distal direction, via the engagement of the sealing member 277 with the barrel 230. To retract the plunger assembly 240, the user must overcome the resistance of the sealing member 277 on the barrel 230 (i.e., Fₚᵤₗₗ>F_{friction}). Although F_{friction} can act in both directions to resist movement of the plunger assembly 240 or keep the plunger assembly 240 fixed when not acted upon, this illustration will strictly define it as the force that opposes the user's pull force Fₚᵤₗₗ.

Referring to FIG. 5A, when vacuum is generated within the barrel 230 (e.g., when the syringe 120 is coupled to the connector 116 of FIG. 1 and the plunger assembly 240 is retracted with the fluid control device 114 of FIG. 1 in the closed position), an additional vacuum force F_{vacuum} acts on the plunger assembly 240 (e.g., the sealing member 277) in the distal direction. To retract the plunger assembly 240 under vacuum, the user must overcome both the resistance of the sealing member 277 on the barrel 230 as well as the opposing vacuum force (i.e., Fₚᵤₗₗ>F_{friction}+F_{vacuum}). The vacuum force F_{vacuum} depends on both the amount of negative pressure generated as well as the cross-sectional area of the plunger assembly 240 subject to the negative pressure (Pressure=Force/Area). When pressure decreases (e.g., negative pressure increases in magnitude) as the plunger assembly 240 is retracted, F_{vacuum} also increases, presenting a larger contribution at the end of the withdrawal stroke. Conversely, as negative pressure approaches its minimum theoretical value (e.g., maximum magnitude), F_{vacuum} plateaus before reaching the end of the withdrawal stroke.

Referring to FIGS. 4A and 5A, the biasing member 285 contributes a biasing force F_{biasing} that acts in the proximal direction against the seal assembly 244 via the coupling of the biasing member 285 against the mount member 284, which is in turn coupled to the second shaft 270. That is, the biasing member 285 biases the mount member 284 proximally through the lumen 252 of the first shaft 250 to urge the second shaft 270 and the sealing head 272 proximally through the barrel 230.

Referring to FIG. 4A, the biasing member 285 can be configured such that the biasing force F_{biasing} is greater than the friction force F_{friction} (i.e., F_{biasing}>F_{friction}). For example, the biasing member 285 can be a compression spring selected to have a spring force (e.g., spring constant) that is greater than the friction force F_{friction}. Accordingly, in the first position shown in FIGS. 4A and 4B in the absence of the vacuum force F_{vacuum} (FIG. 5A), the biasing member 285 is generally rigid and drives/biases the mount member 284 and coupled seal assembly 244 proximally through the barrel 230 such that the locking recesses 405 are positioned with the channels 492 (e.g., as best seen in FIG. 4B). In this first position, the flange 289 of the handle assembly 242 can abut the proximal end portion 493 of the unlocking member 280 and/or the tabs 494 can abut distal end portions of corresponding ones of the channels 492. Accordingly, during retraction of the plunger assembly 240, the plunger assembly 240 acts as an integral plunger in which the handle assembly 242 and the seal assembly 244 move in tandem through the barrel 230 as the biasing member 285 provides a rigid coupling therebetween.

Referring to FIG. 5A, the biasing member 285 can be configured such that the biasing force F_{biasing} is greater than the friction force F_{friction} but less than the combined friction force F_{friction} and vacuum force F_{vacuum} (i.e., F_{vacuum}+F_{friction}>F_{biasing}>F_{friction}). For example, the biasing member 285 can be a compression spring selected to have a spring force (e.g., spring constant) that is greater than the friction force F_{friction} but less than the combined friction force F_{friction} and vacuum force F_{vacuum}. Accordingly, in the second position shown in FIGS. 5A and 5B, the combined friction force F_{friction} and vacuum force F_{vacuum} can overcome the biasing force F_{biasing} of the biasing member 285—causing the biasing member 285 to compress and urge the mount member 284 and coupled seal assembly 244 distally through the barrel 230 such that the locking recesses 405 are spaced apart from and positioned at least partially outside of the channels 492 (e.g., as best seen in FIG. 5B). In this second position, the stop surfaces 499 of the locking features 496 can be spaced apart from the proximal end portion 493 of the unlocking member 280 by a gap G₁ (FIG. 5B).

In some embodiments, after the mount member 284 is drawn distally by the vacuum force F_{vacuum} by a predefined distance (e.g., by a distance equal to the gap G₁ shown in FIG. 5B), the mount member 284 can contact/abut/engage the proximal end portion 286 of the insert member 283 within the lumen 252 of the first shaft 250. This engagement can inhibit or even prevent the mount member 284 and the coupled seal assembly 244 from being drawn farther distally through the barrel 230 by the vacuum force F_{vacuum}. Accordingly, after the plunger assembly 240 reaches the second position shown in FIGS. 5A and 5B, the plunger assembly 240 again acts as an integral plunger in which the handle assembly 242 and the seal assembly 244 move in tandem through the barrel 230 as the insert member 283 rigidly engages the mount member 284. In other embodiments, the mount member 284 need not engage the insert member 283 to fix the plunger assembly 240 in the second position and inhibit further separation between the seal assembly 244 and the handle assembly 242. For example, the biasing member 285 can be configured to fully compress (e.g., bottom out) to inhibit further movement of the seal assembly 244 relative to the handle assembly 242 after a predefined separation between the seal assembly 244 and the handle assembly 242.

Referring to FIGS. 4A and 5A, in some aspects of the present technology the plunger assembly 240 is configured to move between the first and second positions based on the presence of the vacuum force F_{vacuum}. That is, the plunger assembly 240 is vacuum-sensitive. In some embodiments the vacuum force F_{vacuum} is significantly greater than the friction force F_{friction} (e.g., 5 times greater, 10 times greater, 100 times greater, or more; i.e., F_{vacuum}>>F_{friction}). For example, the distal face of the seal assembly 244 can have a relatively large surface area and/or the barrel 230 can have a relatively large volume such that the magnitude of the resultant negative pressure is high. The friction force F_{friction} on the sealing member 277 can be less predictable than the vacuum force F_{vacuum} and can differ as a result of a quantity of silicone applied to the sealing member 277, an amount of compression of the sealing member 277, and/or a length of time since the sealing member 277 was moved. Accordingly, if the vacuum force F_{vacuum} were not much larger than the friction force F_{friction}, it may be difficult to select the biasing member 285 to react to only one force and not the other. Therefore, in some embodiments of the present technology the syringe 120 is configured such that the vacuum force F_{vacuum} is significantly greater than the friction force F_{friction} to make the response of the biasing member 285 more predictable.

Although (i) a discrete first position is shown in FIGS. 4A and 4B in which the seal assembly 244 and the handle assembly 242 are positioned closest to one another (e.g., with the flange 289 of the handle assembly 242 butting the proximal end portion 493 of the unlocking member 280 and/or the tabs 494 abutting distal end portions of corresponding ones of the channels 492) and (ii) a discrete second position is shown in FIGS. 5A and 5B (e.g., with the stop surfaces 499 of the locking features 496 spaced apart from the proximal end portion 493 of the unlocking member 280 by the gap G₁ (FIG. 5B) and the mount member 284 contacting the proximal end portion 286 of the insert member 283), the plunger assembly 240 can have any intermediate position between the first position and the second position depending on, for example, a magnitude of the vacuum force F_{vacuum}. That is, as the vacuum force F_{vacuum} increases (e.g., as the plunger assembly 240 is withdrawn farther proximally through the barrel 230) the vacuum force F_{vacuum} can progressively compress the biasing member 285 to drive the plunger assembly 240 along a continuous spectrum of positions between the first position and the second position. Contrariwise, as the vacuum force F_{vacuum} decreases (e.g., as the plunger assembly 240 is depressed farther distally through the barrel 230 and/or when the fluid control device 114 of FIG. 1 is opened after generating negative pressure in the barrel 230) the energy stored in the biasing member 285 can progressively extend the biasing member 285 to drive the plunger assembly 240 back along the continuous spectrum of positions between the second position and the first position.

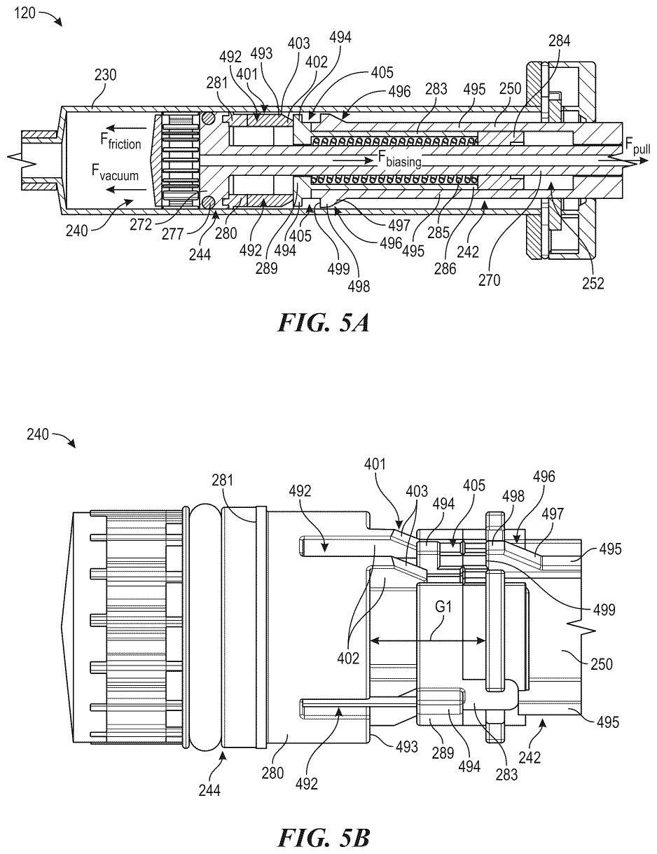

FIG. 6, for example, is an enlarged side view of the distal portion of the plunger assembly 240 shown in FIGS. 4B and 5B in an intermediate position between the first position and the second position in accordance with embodiments of the present technology. In the illustrated embodiment, the stop surfaces 499 of the locking features 496 are spaced apart from the proximal end portion 493 of the unlocking member 280 by a gap G₂ smaller than the gap G₁ (FIG. 5B) in the second position. Similarly, the locking recesses 405 and the lock features 496 are at least partially exposed from and uncovered (e.g., not radially shielded) by the channels 492 in the unlocking member 280. As further shown in FIG. 6, the insert member 283 can include a flange 609 extending at least partially circumferentially thereabout and defining a distal surface 609. The distal surface 609 can be longitudinally aligned with the stop surfaces 499 of the lock features 496. Accordingly, the gap G₂ can be defined between the flange 609 and the proximal end portion 493 of the unlocking member 280.

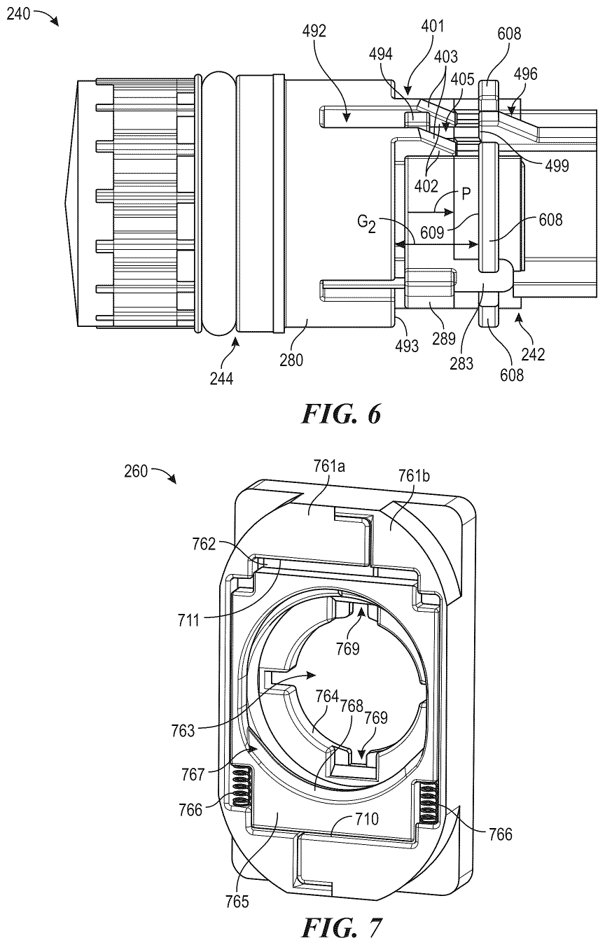

FIG. 7 is an isometric view of the locking assembly 260 of the syringe 120 of FIGS. 2A and 2B in accordance with embodiments of the present technology. In the illustrated embodiment, the locking assembly 260 includes a housing 761 (including an individually identified first housing portion 761a and a second housing portion 761b) defining a recess 762 and having a housing opening 763 extending axially therethrough and defined by an edge portion 764. In some embodiments, the housing 761 comprises multiple portions that can be interlocked or otherwise coupled together, such as the first housing portion 761a, the second housing portion 762b, and a third housing portion 762c (omitted in FIG. 7 for clarity; shown in FIG. 2B). Referring to FIGS. 2B and 7, the housing 761 can be assembled by interlocking or otherwise coupling the first housing portion 761a and the second housing portion 761b to define the recess 762 and the housing opening 763, positioning the flange 231 of the barrel 230 against the first and second housing portions 761a-b, and then coupling the third housing portion 761c over the flange 231 and to the first and second housing portions 761a-b. In other embodiments, the housing 761 can have a different shape, can be coupled to the barrel 230 in different manners, can comprise a different number of discrete portions, and/or can be integrally formed.

Referring to FIG. 7, the locking assembly 260 further includes a lock member or lock plate 765 coupled to the housing 761 within the recess 762 via one or more biasing members 766 (e.g., a pair of the biasing members 766). The lock plate 765 can include a lock plate opening 767 extending axially therethrough and defined by an edge including a lock rail 768 (which can also be referred to as a locking edge, a locking edge portion, a plunger engagement portion, and/or the like). Referring to FIGS. 2A, 4A-5B, and 7, the locking assembly 260 can receive the plunger assembly 240 through the housing opening 763 and the lock plate opening 767. In some embodiments, the edge portion 764 of the lock plate 765 defining the housing opening 764 can include one or more channels 769 (e.g., cutouts, grooves) configured (e.g., shaped, sized, positioned) to slidingly receive one or more of the rails 495 of the first shaft 250 of the plunger assembly 240 therethrough to, for example, facilitate and maintain alignment of the plunger assembly 240, more specifically the lock feature 496 and the unlock feature 401, relative to the locking assembly 260.

Referring to FIG. 7, the recess 762 can have a first end portion 710 and a second end portion 711 opposite the first end portion 710. The biasing members 766 can extend between and operably couple the lock plate 765 to the housing 761 proximate the first end portion 710 of the recess 762. The biasing members 766 can comprise, for example, compression springs that bias the lock plate 765 away from the first end portion 710 of the recess 762 toward the second end portion 711 of the recess 762. The locking assembly 260 is shown in a first position (e.g., a compressed position) in FIG. 7 in which the lock plate 765 is urged against the force of the biasing members 766 toward the first end portion 710 of the recess 762. In some embodiments, in the first position, the lock plate 765 can (i) abut the housing 761 at the first end portion 710 of the recess 762 and (ii) be spaced apart from the housing 761 at the second end portion 711 of the recess 762. Absent a force against the lock plate 765, the biasing members 766 can drive the lock plate 765 through the recess 762 to a second position (e.g., an extended position, a relaxed position) in which the lock plate 765 is urged toward the second end portion 711 of the recess 762. That is, the lock plate 765 can be normally biased to the second position. In some embodiments, in the second position, the lock plate 765 can (i) abut the housing 761 at the second end portion 711 of the recess 762 and (ii) be spaced apart from the housing 761 at the first end portion 710 of the recess 762.

FIGS. 8A and 8B are enlarged cross-sectional side views of the syringe 120 with the plunger assembly 240 proximally withdrawn through the barrel 230 to a withdrawn position and locked to the locking assembly 260 and unlocked from the locking assembly 260, respectively, in accordance with embodiments of the present technology. Referring to FIG. 8A, the plunger assembly 240 is in the first position described in detail with reference to FIGS. 5A and 5B in which the vacuum force F_{vacuum} actuates the plunger assembly 240 such that (i) the flange 289 of the handle assembly 242 is spaced apart from the proximal end portion 493 of the unlocking member 280 by the gap G₁ (FIG. 5B) and (ii) the locking recesses 405 are spaced apart from and uncovered (e.g., not radially shielded) by the unlocking member 280. As the plunger assembly 240 is withdrawn proximally through the barrel 230 with the plunger assembly 240 in the first position, the lock feature 496 is spaced apart from and exposed from the unlocking member 280 (e.g., spaced apart from the unlock feature 401).

During withdrawal of the plunger assembly 240, the lock feature 496 is configured to engage the lock rail 768 of the lock plate 765 of the locking assembly 260 to move the lock plate 765 from the normally-biased second position toward the first position. More specifically, the sloped ramp portion 497 of the lock feature 496 can engage the lock rail 768 and drive the lock rail 768 and lock plate 765 toward the first end portion 710 of the recess 762 in the housing 761 until the lock plate 765 reaches the plateau portion 498 of the lock feature 496. That is, the ramp portion 497 of the lock feature 496 translates the longitudinal force of the handle assembly 242 during withdrawal into radial movement of the lock plate 765. Continued withdrawal of the plunger assembly 240 slides the lock rail 768 of the lock plate 765 over/against the plateau portion 498 until the lock rail 768 reaches the stop surface 499. When the lock rail 768 passes the end of the plateau portion 498, the biasing members 766 (FIG. 7) can drive the lock plate 765 toward the second end portion 711 of the recess 762 (e.g., from the compressed first position toward the second position). At this stage, as shown in FIG. 8A, the lock rail 768 of the lock plate 765 is positioned within the locking recess 405 formed by the tab 494 and the lock feature 496 such that plunger assembly 240 is locked in position along the longitudinal axis L (FIG. 2A) relative to the locking assembly 260 (e.g., inhibited from moving/advancing past the lock plate 765 toward the depressed position shown in FIG. 2A). In some embodiments, the lock rail 768 can be positioned distal of the distal surface 609 of the flange 608 (obscured in FIGS. 8A and 8B; FIG. 6). Accordingly, the distal surface 609 of the flange 608 and/or the stop surface 499 of the lock feature 496 can abut the lock rail 768 to inhibit or even prevent distal movement of the plunger assembly 240. In some embodiments, the tab 494 can be omitted as the lock plate 765 inhibits distal movement of the plunger assembly 240 and the housing 761 controls (e.g., inhibits or even prevents) further proximal movement of the plunger assembly 240.

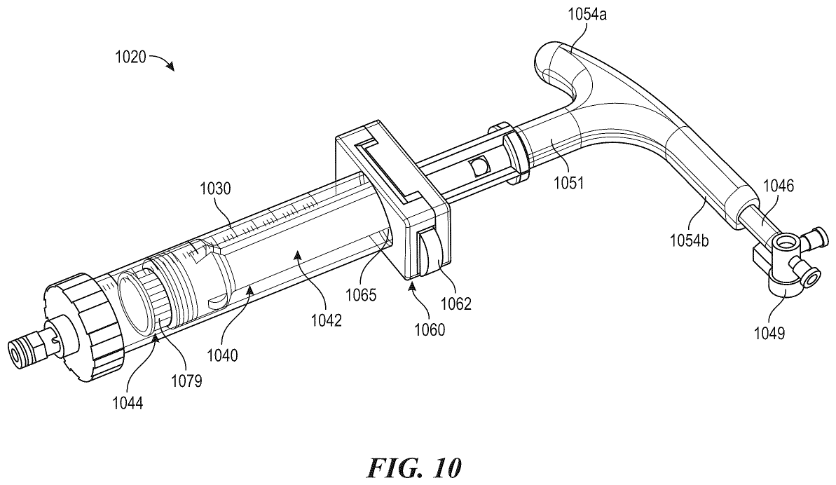

The plunger assembly 240 can remain locked to the locking assembly 260 so long as the vacuum force F_{vacuum} remains and maintains the plunger assembly 240 in the first position shown in FIG. 8A. When the vacuum is released or the vacuum force F_{vacuum} falls below the biasing force F_{biasing}, the seal assembly 244 of the plunger assembly 240 can automatically move from the first position shown in FIG. 8A to the second position shown in FIG. 8B to unlock the plunger assembly 240 from the locking assembly 260. More specifically, when the vacuum force F_{vacuum} approaches zero, the biasing force F_{biasing} of the biasing member 285 can drive the mount member 284 proximally through the first shaft 250 to pull the unlocking member 280 proximally toward the locking recess 405 of the handle assembly 242. As best seen in FIG. 6, as the unlocking member 280 moves proximally in the direction of arrow P, the unlock feature 401 can slide past the locking recess 405 as the tab 494 and the lock feature 496 slide through and into the channel 492. Referring to FIGS. 6 and 8B, similar to the operation of the ramp portion 497 of the lock feature 496, the proximal movement of the unlocking member 280 drives the ramp portions 403 (FIG. 6) of the projections 402 (FIG. 6) of the unlocking member 280 against the lock rail 768 of the lock plate 765 to move the lock plate 765 from the normally-biased second position toward the first position. More specifically, the sloped ramp portions 403 of the unlock feature 401 engage the lock rail 768 and drive the lock rail 768 and lock plate 765 toward the first end portion 710 of the recess 762 in the housing 761 and out of the locking recess 405. When the lock plate 765 is removed from the locking recess 405 as shown in FIG. 8B, the plunger assembly 240 is unlocked from the locking assembly 260 and can be moved (e.g., depressed through the barrel 230). In some embodiments, the locking assembly 260 can include a button or other feature that is actuatable to manually remove the lock rail 768 from the locking recess 405 even when there is little or no vacuum within the barrel 230, as described in greater detail below with reference to FIG. 10.

Referring to FIGS. 8A and 8B, only one of the locking recesses 405 and one of the lock features 496 at a lower side of the syringe 120 is configured to engage the lock plate 765. Accordingly, the syringe 120 can include only one of the locking recesses 405 and one of the lock features 496 positioned to engage the lock plate 765. In the illustrated embodiment, the one of the locking recesses 405 and the one of the lock features 496 at the upper side of the syringe 120 can provide for redundancy of installation of the plunger assembly 240 within the barrel 230 and the locking assembly 260—for example, allowing the plunger assembly 240 to be installed in a flipped or rotated (e.g., by 180 degrees) configuration without loss of functionality.

Referring to FIGS. 2A-8B together, in some aspects of the present technology the syringe 120 is configured to (i) automatically lock the plunger assembly 240 to the locking assembly 260 when the plunger assembly 240 is withdrawn through the barrel 230 with a vacuum within the barrel 230, (ii) automatically unlock the plunger assembly 240 from the locking assembly 260 when the barrel 230 no longer experiences vacuum, and (iii) inhibit or even prevent automatic locking of the plunger assembly 240 to the locking assembly 260 when the plunger assembly 240 is withdrawn through the barrel 230 with no or negligible vacuum within the barrel 230. For example, regarding (i) and as described in detail above with reference to FIG. 8A, the vacuum force F_{vacuum} acting on the plunger assembly 240 can overcome the biasing force F_{biasing} of the biasing member 285 to move the unlocking member 280 away from the locking recess 405 to expose the locking recess 405 such that the lock plate 765 can be driven into the locking recess 405 (e.g., via the lock feature 496) to lock the position of the plunger assembly 240 relative to the locking assembly 260. Regarding (ii) and as described in detail above with reference to FIG. 8B, release of the vacuum force F_{vacuum} permits the biasing force F_{biasing} of the biasing member 285 to drive the unlocking member 280 to engage the lock plate 765 and drive the lock plate 765 out of the locking recess 405 (e.g., via the unlock feature 401) to unlock the position of the plunger assembly 240 relative to the locking assembly 260. Finally, regarding (iii), in the absence of the vacuum force F_{vacuum} the unlocking member 280 surrounds and radially shields (e.g., covers) the locking recess 405 such that the lock plate 765 cannot be inserted into the locking recess 405 even as the plunger assembly 240 is withdrawn through the locking assembly 260.

Accordingly, the syringe 120 can be an automatic-locking and automatic-unlocking syringe 120 in response to vacuum pressure in the barrel 230. In contrast, some conventional syringes may automatically lock upon withdrawal of a plunger or be user-actuatable to selectively lock the position of the plunger. However, these devices require a user input to lock the plunger and/or to unlock the plunger and allow its further movement. In some aspects of the present technology, the automatic-locking and automatic-unlocking syringe 120 of the present technology can simplify a clot removal procedure using the syringe 120. For example, referring to FIGS. 1-2B, during a clot removal procedure using the system 100, the plunger assembly 240 can be withdrawn with the fluid control device 114 in a closed position to generate vacuum pressure in the barrel 230 that acts on the plunger assembly 240 to allow the plunger assembly 240 to lock in position to the lock plate 765 without any user (e.g., physician) input when fully withdrawn through the barrel 230. The fluid control device 114 can then be opened to apply the vacuum pressure stored in the barrel 230 to the catheter 106 to aspirate clot material and blood into the barrel 230. With the vacuum pressure released, the plunger assembly 240 can automatically unlock from the locking assembly 260 without user input via the biasing member 285 urging the unlock feature 401 against the lock plate 765. With the fluid control device 114 closed, the plunger assembly 240 can then be depressed through the barrel 230 to drive the blood through the filter 279 and the filtered blood out through the outlet tube 246 (and/or the outlet tube 246 may be aspirated to draw the blood through the filter 279 and the filtered blood out through the outlet tube 246). Such automatic locking and unlocking of the syringe 120 simplifies the clot removal procedure.

In additional aspects of the present technology, the syringe 120 can act as a normal syringe (e.g., without automatic locking) when vacuum is not generated within the barrel 230. For example, when the syringe 120 is used for procedural steps other than aspiration, such as contrast injection, saline flushing, and/or the like, the syringe 120 can act as a standard syringe that does not lock to the locking assembly 260 when the plunger assembly 240 is withdrawn during a stroke. More specifically, when little or no vacuum pressure is generated within the barrel 230 during withdrawal of the plunger assembly 240, the biasing member 285 can bias the seal assembly 244 to the first position shown in FIGS. 4A and 4B in which the tabs 494 and the locking features 496 of the handle assembly 242 are positioned within the corresponding ones of the channels 492 formed in/by the unlocking member 280 and the unlock features 401 such that the locking recesses 405 are covered (e.g., radially shielded) by the unlocking member 280. Accordingly, when the plunger assembly 240 is withdrawn through/past the locking assembly 260 the lock plate 765 does not engage the locking recesses 405 and the plunger assembly 240 can be freely withdrawn proximally and/or advanced distally without locking to the locking assembly 260.

Referring to FIG. 2A, in some embodiments the syringe 120 can include an indicator 222 configured to provide an indication to the user of a level of vacuum in the barrel 230 and/or in the system 100 (FIG. 1) as a whole. In the illustrated embodiment, the indicator 222 includes an (i) aperture or window 223 in the first shaft 250 of the handle assembly 242 and (ii) a visual indicator 224 on the second shaft 270 and at least partially visible through the window 223. As described in detail above with reference to FIGS. 4A-6, the second shaft 270 translates within the first shaft 250 as the seal assembly 244 moves relative to the handle assembly 242 in response to vacuum pressure within the barrel 230. This movement can position the visual indicator 224 differently with respect to the window 223 based on a level of vacuum in the barrel 230.

Referring to FIG. 3, the visual indicator 224 can comprise a color scale including, for example, a first color 225 (e.g., red) along a first portion of the second shaft 270, a second color 226 (e.g., orange) along a second portion of the second shaft 270 proximal of the first portion, and a third color 227 (e.g., green) along a third portion of the second shaft 270 proximal of the second portion. Referring to FIGS. 2A and 3, when vacuum is not present in the barrel 230 or there is negligible vacuum within the barrel 230, the plunger assembly 240 is in the first position as shown in FIGS. 2A and 3 and the first color 225 can be visible through the window 223 indicating that there is no or negligible vacuum within the barrel 230. When a maximum or substantially maximum vacuum is within the barrel 230, the plunger assembly 240 is in the second position shown in FIGS. 5A and 5B and the third color 227 can be visible through the window 223 indicating that there is the maximum or substantially maximum vacuum within the barrel 230. When an intermediate vacuum between no vacuum and the maximum vacuum is within the barrel 230, the plunger assembly 240 is in an intermediate position (e.g., the intermediate position shown in FIG. 6) and the second color 226 can be visible through the window 223 indicating that there is the intermediate vacuum within the barrel 230. Likewise, when the plunger assembly 240 is in a withdrawn position, the first color 225 can indicate that the plunger assembly 240 is unlocked from the locking assembly 260, the second color 226 can indicate that plunger assembly 240 is partially unlocked, in the process of unlocking, and/or will soon unlock from the locking assembly 260, and the third color 227 can indicate that the plunger assembly 240 is locked to the locking assembly 260. In other embodiments, rather than different colors, the visual indicator 224 can comprise different icons, textures, values, marks, etc., that indicate the various vacuum and locking states.

FIG. 9 is a table including various views of the syringe 120 at different vacuum levels in accordance with embodiments of the present technology. More specifically, referring to FIGS. 2A-9, the views in the first column show the syringe 120 under no or negligible vacuum (e.g., less than 25% of a 100% maximum vacuum), the views in the second column show the syringe 120 under 50% of the maximum vacuum, the views in the third column show the syringe 120 under 75% of the maximum vacuum, and the views in the fourth column show the syringe 120 under the 100% maximum vacuum. The views in row A are partially-transparent side views of the syringe 120 proximate the locking assembly 260 with the plunger assembly 240 withdrawn to near the position at which the plunger assembly 240 can lock the locking assembly 260 (e.g., with the locking recesses 405 of the handle assembly 242 and/or the unlocking member 280 of the seal assembly 244 proximate to the lock plate 765). The views in row B are cross-sectional side views of a proximal portion of the syringe 120 at and proximal of the locking assembly 260 with the plunger assembly 240 withdrawn to near the position at which the plunger assembly 240 can lock the locking assembly 260. The views in row C are side views of a proximal portion of the handle assembly 242 including the indicator 222.

FIG. 10 is an isometric view of a syringe 1020 in accordance with additional embodiments of the present technology. The syringe 1020 can include some features that are at least generally similar in structure and function, or identical in structure and function, to the corresponding features of the syringe 120 described in detail above with reference to FIGS. 1-9, and can operate in a generally similar or identical manner to the syringe 120. For example, in the illustrated embodiment the syringe 1020 is an automatic-locking and automatic-unlocking syringe including a plunger assembly 1040 slidably positioned within a barrel 1030. The barrel 1030 is shown as partially transparent in FIG. 10 for clarity. In the illustrated embodiment, the plunger assembly 1040 includes a handle assembly 1042 operably coupled to a seal assembly 1044, and a locking assembly 1060 coupled to the barrel 1030. When the plunger assembly 1040 is withdrawn through the barrel 1030 and vacuum pressure is generated within the barrel 1030, the seal assembly 1044 is configured to move away from the handle assembly 1042 to permit locking of the plunger assembly 1040 to the locking assembly 1060 as described in detail above with reference to FIGS. 1-9.

The handle assembly 1042 includes a proximal end portion 1051 configured (e.g., shaped, sized) to be grasped by a user for withdrawing (e.g., retracting, pulling) and/or depressing (e.g., advancing, pushing) the plunger assembly 1040 through the barrel 1030 during operation of the syringe 1020. For example, in the illustrated embodiment the proximal end 1051 includes a pair of opposing flanges 1054 (individually identified as a first flange 1054a and a second flange 1054b) extending radially outward from a longitudinal axis of the syringe 1020 and forming a handle configured to be grasped by a hand of the user. In some embodiments, the second flange 1054b is larger than the first flange 1054a. An outlet tube 1046 can extend at least partially through the handle assembly 1042 and through the second flange 1054b to a connector 1049, such as a multi-lumen connector 1049. The seal assembly 1044 can include a filter 1079 fluidly coupled to the outlet 1046 via, for example, a lumen extending at least partially through the seal assembly 1044 (e.g., the lumen in the second shaft 270 described in detail above with reference to FIGS. 2A and 2B). The connector 1049 can be coupled to a reservoir/container for receiving blood filtered by the filter 1079 (e.g., as the plunger assembly 1040 is depressed with clot material and blood in the barrel 1030), a fluid source for injecting fluid into the barrel 1030, and/or the like.

In the illustrated embodiment, the locking assembly 1060 further includes an actuator 1062 operably coupled to a lock plate 1065 (partially obscured in FIG. 10). The lock plate 1065 can engage the plunger assembly 1040 (e.g., a locking recess formed thereby/therein) to selectively lock the plunger assembly 1040 relative to the barrel 1030 as described in detail above with reference to FIGS. 2A-9. The actuator 1062 can comprise a button and/or the like configured to be actuated (e.g., depressed) by a user to manually unlock the lock plate 1065 from the plunger assembly 1040 to, for example, allow the plunger assembly 1040 to move (e.g., be depressed) through the barrel 1030. In some aspects of the present technology, the actuator 1062 can act as an override feature to allow the plunger assembly 1040 to be unlocked from the locking assembly 1060 when there is significant vacuum pressure within the barrel 1030. In addition to manual unlock from the locking assembly 1060 via the actuator 1062, the plunger assembly 1040 can be configured to automatically unlock from the locking assembly 1060 as described in detail above with reference to FIGS. 2A-9. In some embodiments, the locking assembly 1060 and the actuator 1062 can include some features generally similar in structure and/or function, or identical in structure and/or function, to those of any of the locking assemblies and/or actuators (e.g., buttons) described in U.S. patent application Ser. No. 17/396,426, titled “AUTOMATICALLY-LOCKING VACUUM SYRINGES, AND ASSOCIATED SYSTEMS AND METHODS,” and filed Aug. 8, 2021, which is incorporated herein by reference in its entirety.

FIGS. 11A and 11B are side views of a syringe 1120 that can be used in the clot treatment system of FIG. 1 in a first position (e.g., a depressed position) and a second position (e.g., a withdrawn position), respectively, in accordance with additional embodiments of the present technology. Referring to FIGS. 11A and 11B, the syringe 1120 can include some features that are at least generally similar in structure and function, or identical in structure and function, to the corresponding features of the syringe 120 and/or the syringe 1020 described in detail above with reference to FIGS. 1-10, and can operate in a generally similar or identical manner to the syringe 120 and/or the syringe 1020. For example, in the illustrated embodiment the syringe 1120 is an automatic-locking and automatic-unlocking syringe including a plunger assembly 1140 slidably positioned within a barrel 1130.

In the illustrated embodiment, the plunger assembly 1140 includes a handle assembly 1142 operably coupled to a seal assembly 1144. The syringe 1120 further includes a locking assembly 1160 coupled to the barrel 1130. The locking assembly 1160 is configured to selectively lock the plunger assembly 1140 in a withdrawn position under vacuum pressure as described in detail above with reference to FIGS. 8A-10. When the plunger assembly 1140 is withdrawn through the barrel 1130, as shown in FIG. 11B, and vacuum pressure is generated within the barrel 1130, the seal assembly 1144 is configured to move away from the handle assembly 1142 to permit locking of the plunger assembly 1140 to the locking assembly 1160 as described in detail above with reference to FIGS. 1-10. Referring to FIG. 11A, the plunger assembly 1140 is in the first position and is fully depressed within the barrel 1130. Referring to FIG. 11B, the plunger assembly 1140 is in the second position and is withdrawn through the barrel 1130 and locked to the locking assembly 1160. In the illustrated embodiment, the syringe 1120 is not a filtering syringe and, for example, the seal assembly 1144 does not include features such as the filter 279, the cap 278, and/or the distal opening 274 described in detail above with reference to FIGS. 2A and 2B. Accordingly, the handle assembly 1142 need not define a lumen therethrough for, for example, receiving filtered blood.

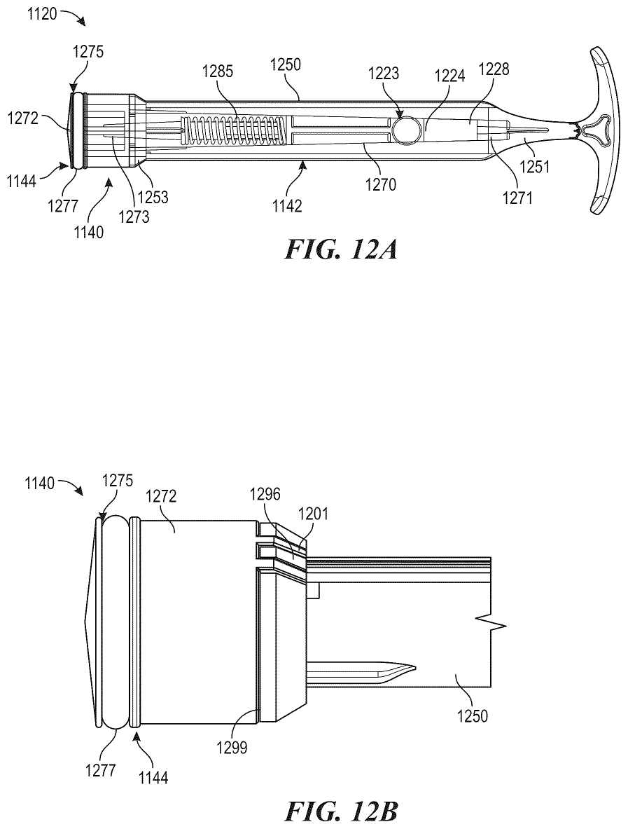

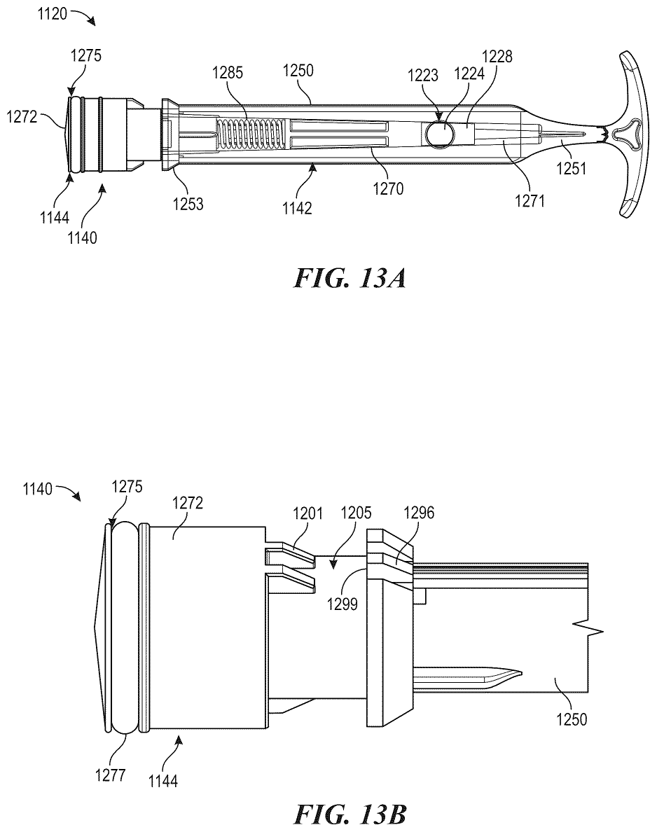

FIGS. 12A and 13A are partially-transparent side views of the plunger assembly 1140 of the syringe 1120 of FIGS. 11A and 11B in a first position (e.g., a non-vacuum position) and a second position (e.g., a vacuum position), respectively, in accordance with embodiments of the present technology. Referring to FIGS. 12A and 13A, in the illustrated embodiment the handle assembly 1142 includes a first shaft 1250 extending between a proximal end portion 1251 and a distal end portion 1253. The proximal end portion 1251 can be configured to be grasped by a user for withdrawing and/or depressing the plunger assembly 1140 through the barrel 1130 during operation of the syringe 1120, as described in detail above with reference to FIGS. 1-10. The seal assembly 1144 includes a second shaft 1270 having a proximal end portion 1271 and a distal end portion 1273 (FIG. 12A). The distal end portion 1273 of the second shaft 1270 can be coupled to a sealing head 1272. A circumferential groove 1275 in the sealing head 1272 can receive a sealing member 1277 therein, such as an O-ring, configured to sealingly engage an interior surface of the barrel 1130 (FIGS. 11A and 11B).

The plunger assembly 1140 further includes a biasing member 1285 operably coupling the first shaft 1250 to the second shaft 1270. The handle assembly 1142 can be configured to be withdrawn relative to the barrel 1130 (FIGS. 11A and 11B) to withdraw the seal assembly 1244 and the sealing member 1277 through the barrel 1130 to generate vacuum pressure within the barrel 1130. The vacuum pressure can generate a vacuum force on the seal assembly 1144 greater than a biasing force of the biasing member 1285 such that the biasing member 1285 compresses and the sealing head 1272 moves away from the distal portion of the first shaft 1250 as shown in FIG. 13A.

FIGS. 12B and 13B are enlarged side views of a distal portion of the plunger assembly 1140 of FIGS. 12A and 13A, respectively, in accordance with embodiments of the present technology. Referring to FIGS. 12B and 13B, in the illustrated embodiment the sealing head 1272 is configured as an unlocking member (e.g., an integral component integrating/combining, for example, the sealing head 272 and the unlocking member 280 of FIGS. 2A and 2B). In the illustrated embodiment, the sealing head 1272 includes one or more unlock features 1201 and the first shaft 1250 includes one or more lock features 1296. The lock features 1296 are aligned with corresponding ones of the unlock features 1201 and configured to nest therein in the first position (FIG. 12B) with a stop surface 1299 of the locking features 1296 abutting the sealing head 1272. In the second position (FIG. 13B), vacuum pressure on the sealing head 1272 compresses the biasing member 1285 (FIGS. 12A and 13A) such that the lock features 1296 are spaced apart from the unlock features 1201 to define a locking recess 1205. In some embodiments, compared to the embodiment of FIGS. 2-9, the plunger assembly 1120 can include a configuration of tabs (not shown; e.g., the tabs 494 of FIGS. 4B-6) and channels (not shown; e.g., the channels 492 of FIGS. 4B-6) that are internal to the first shaft 1250 and sealing head 1272 to facilitate continued alignment of these components.

Referring to FIGS. 12A and 13A, the plunger assembly 1140 can include an aperture or window 1223 in the first shaft 1250 and the second shaft 1270 of the handle assembly 1142 configured to provide an indication to the user of a level of vacuum in the barrel 1130 and/or in the system 100 (FIG. 1) as a whole. As described in detail above with reference to FIGS. 4A-6, the second shaft 1270 translates within the first shaft 1250 as the seal assembly 1144 moves relative to the handle assembly 1142 in response to vacuum pressure within the barrel 1130. This movement can position a visual indicator 1224 differently with respect to the window 1223 based on a level of vacuum in the barrel 1130 (FIGS. 11A and 11B). In other embodiments, the aperture or window 1223 can comprise different-shaped openings in the first shaft 1250 and the second shaft 1270 of the handle assembly 1142.

The visual indicator 1224 can comprise a solid color 1228. Referring to FIG. 12A, when vacuum is not present in the barrel 1130 or there is negligible vacuum within the barrel 1130, the plunger assembly 1140 is in the first position and the solid color 1228 is not visible through the window 1223, indicating that there is no or negligible vacuum within the barrel 1130. When vacuum is generated within the barrel 1130, the plunger assembly 1140 is in the second position and the solid color 1228 can be visible through the window 1223 indicating that there is vacuum pressure within the barrel 1130. Likewise, when the plunger assembly 1140 is in a withdrawn position, the lack of visibility of the solid color 1228 can indicate that the plunger assembly 1140 is unlocked from the locking assembly 1160, and the visibility of the solid color 1228 can indicate that the plunger assembly 1140 is locked to the locking assembly 1160. For example, the visual indicator 1224 is visible through the window 1223 in FIG. 11B indicating that the plunger assembly 1140 is locked to the locking assembly 1160. In other embodiments, rather than different colors, the visual indicator 1224 can comprise different icons, textures, values, marks, etc., that indicate the various vacuum and locking states.

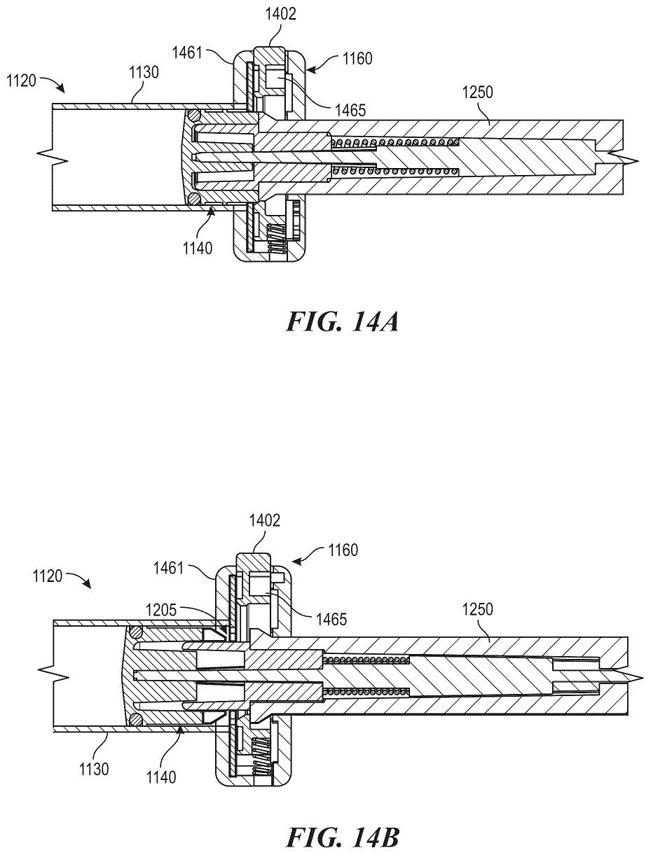

FIGS. 14A and 14B are cross-sectional side views of the syringe 1120 of FIGS. 11A and 11B in an unlocked position and a locked position, respectively, in accordance with additional embodiments of the present technology. Referring to FIGS. 14A and 14B, in the illustrated embodiment the locking assembly 1160 includes a housing 1461 and a lock plate 1465. In the locked position shown in FIG. 14B, the plunger assembly 1140 is in the second position (FIGS. 12B and 13B) and the lock plate 1465 can engage the plunger assembly 1140 (e.g., the locking recess 1205 formed thereby/therein) to selectively lock the plunger assembly 1140 relative to the barrel 1130. In the unlocked position shown in FIG. 14A, the plunger assembly 1140 is in the first position (FIGS. 12A and 13A) such that the locking recess 1205 is shielded and the lock plate 1465 does not engage the locking recess 1205. A button portion 1402 of the lock plate 1465 can protrude from the housing 1461 when the locking assembly 1160 is in the locked position. The button portion 1402 is configured to be actuated (e.g., depressed) by a user to manually unlock the lock plate 1465 from the plunger assembly 1140 to, for example, allow the plunger assembly 1140 to move (e.g., be depressed) through the barrel 1130. In some aspects of the present technology, the lock plate 1465 (e.g., the button portion 1402) can act as an override feature to allow the plunger assembly 1140 to be unlocked from the locking assembly 1160 when there is significant vacuum pressure within the barrel 1130.

In addition to manual unlock from the locking assembly 1160 via the lock plate 1465, the plunger assembly 1140 can be configured to automatically unlock from the locking assembly 1160 as described in detail above with reference to FIGS. 2A-9. For example, when vacuum pressure is released, the biasing member 1285 can move the sealing head 1272 toward the first shaft 1250 such that the ramped unlock features 1201 drive the lock plate 1465 out of the locking recess 1205. In some embodiments, the locking assembly 1160 and the lock plate 1465 can include some features generally similar in structure and/or function, or identical in structure and/or function, to those of any of the locking assemblies and/or actuators (e.g., buttons) described in U.S. patent application Ser. No. 17/396,426, titled “AUTOMATICALLY-LOCKING VACUUM SYRINGES, AND ASSOCIATED SYSTEMS AND METHODS,” and filed Aug. 8, 2021, which is incorporated herein by reference in its entirety.

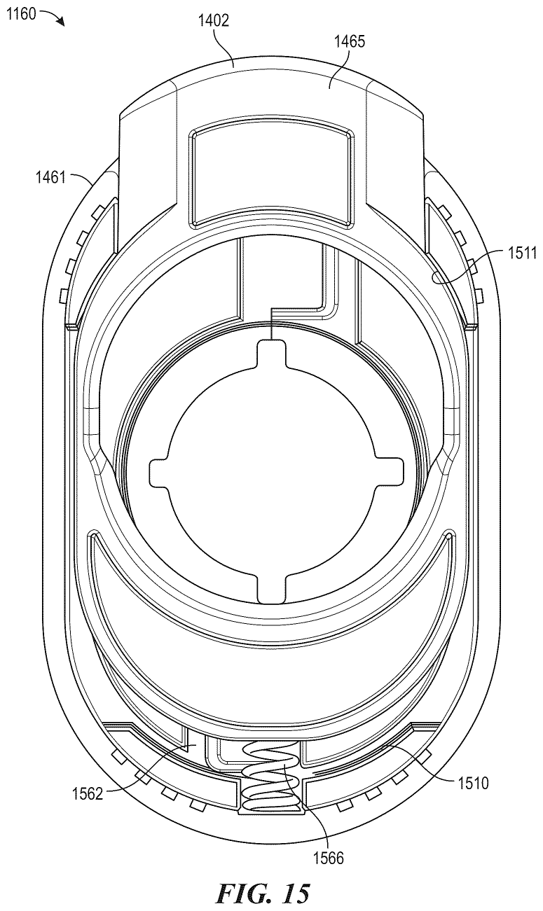

FIG. 15 is a cross-sectional view of the locking assembly 1160 of the syringe 1120 of FIGS. 11A and 11B in accordance with embodiments of the present technology. In the illustrated embodiment, the housing 1461 defines a recess 1562 having a first end portion 1510 and a second end portion 1511 opposite the first end portion 1510. One or more biasing members 1566 can extend between and operably couple the lock plate 1465 to the housing 1461 proximate the first end portion 1510 of the recess 1562. The biasing member 1566 can comprise, for example, a compression spring that biases the lock plate 1465 away from the first end portion 1510 of the recess 1562 toward the second end portion 1511 of the recess 1562. Referring to FIGS. 14A-15 together, in the locked position (FIG. 14B) the biasing member 1566 forces the lock plate 1465 into the locking recess 1205. When vacuum pressure is released within the barrel 1130 the ramped unlock features 1201 engage the lock plate 1465 to move the lock plate 1465 against the biasing force of the biasing member 1566 to drive the lock plate 1465 out of the locking recess 1205 as shown in FIG. 14A. Likewise, actuation of the button portion 1402 can drive the lock plate 1465 against the biasing force of the biasing member 1566 to release the lock plate 1465 from the locking recess 1205 to unlock the plunger assembly 1140 from the locking assembly 1160.

The following examples are illustrative of several embodiments of the present technology:
1. An automatic-locking and automatic-unlocking syringe, comprising:
   a barrel;
   a plunger assembly slidably positioned within the barrel, wherein the plunger assembly is movable between a first position and a second position; and
   a locking assembly coupled to the barrel, wherein—
      the plunger assembly is configured to be withdrawn through the barrel to generate vacuum pressure in the barrel;
      the plunger assembly is configured to move from the first position to the second position in response to the vacuum pressure;
      the plunger assembly is configured to move from the second position to the first position in response to the vacuum pressure being released;
      the plunger assembly is configured to lock to the locking assembly in the second position such that the locking assembly inhibits movement of the plunger assembly through the barrel; and
      the plunger assembly is configured to unlock from the locking assembly in the first position such that the locking assembly permits movement of the plunger assembly through the barrel.
2. The syringe of example 1 wherein the plunger assembly includes a sealing head, a shaft, and a biasing member operably coupling the sealing head to the shaft.
3. The syringe of example 2 wherein in the first position the biasing member biases the sealing head into engagement within the shaft.
4. The syringe of example 3 wherein in the second position the vacuum pressure moves the sealing head from the shaft, against a biasing force of the biasing member, to define a locking recess therebetween.
5. The syringe of example 4 wherein the locking assembly includes a lock plate configured to be positioned in the locking recess when the plunger assembly is in the second position such that the lock plate inhibits movement of the plunger assembly through the barrel.
6. The syringe of example 5 wherein movement of the plunger assembly from the second position to the first position is configured to drive the lock plate from the locking recess to permit movement of the plunger assembly through the barrel.
7. The syringe of example 5 or example 6 wherein the lock plate includes a button portion configured to be actuated by a user to drive the lock plate out of the locking recess when the plunger assembly is in the second position to permit movement of the plunger assembly through the barrel.
8. The syringe of any one of examples 1-7 wherein the locking assembly includes a housing, a lock plate positioned at least partially within the housing, and a biasing member operably coupling the lock plate to the housing.
9. The syringe of example 8 wherein the biasing member is configured to drive the lock plate into locking engagement with the plunger assembly when the plunger assembly is in the second position, and wherein the plunger assembly is configured to drive the lock plate against a biasing force of the biasing member when the plunger assembly is in the first position.
10. The syringe of any one of examples 1-9 wherein the plunger assembly further comprises a filtering seal assembly configured to separate clot material from blood.
11. A syringe, comprising:
   a barrel; and
   a plunger assembly slidably positioned within the barrel, wherein the plunger assembly comprises—
      a handle assembly including a first shaft having a distal portion;
      a seal assembly including a second shaft and a seal head coupled to the second shaft, wherein the seal head includes a sealing member positioned to sealingly engage the barrel; and
      a biasing member operably coupling the first shaft to the second shaft, wherein the biasing member has a biasing force, and wherein—
         the handle assembly is configured to be withdrawn relative to the barrel to withdraw the seal assembly and the sealing member through the barrel to generate vacuum pressure within the barrel; and
         the vacuum pressure is configured to generate a vacuum force on the seal assembly greater than the biasing force of the biasing member such that the biasing member compresses and the seal head moves away from the distal portion of the first shaft.
12. The syringe of example 11 wherein the biasing member is a compression spring.
13. The syringe of example 11 or example 12 wherein in the absence of the vacuum pressure the biasing force is configured to drive the seal head to engage the distal portion of the first shaft.
14. The syringe of any one of examples 11-13, further comprising a locking assembly including a lock plate wherein the vacuum pressure is configured to generate the vacuum force on the seal assembly greater than the biasing force of the biasing member such that the seal head moves away from the distal portion of the first shaft to define a locking recess therebetween, and wherein the lock plate is configured to be positioned in the locking recess to inhibit movement of the plunger assembly through the barrel.
15. The syringe of example 14 wherein in the absence of the vacuum pressure the biasing force is configured to drive the seal head to engage the distal portion of the first shaft to drive the lock plate from the locking recess.
16. The syringe of any one of examples 11-15 wherein the second shaft comprises a visual indicator, wherein the first shaft includes an aperture, and wherein the visual indicator is at least partially visible through the aperture when the vacuum force on the seal assembly is greater than the biasing force of the biasing member such that the seal head moves away from the distal portion of the first shaft.
17. A syringe, comprising:
   a barrel; and
   a plunger assembly slidably positioned within the barrel, wherein the plunger assembly comprises—
      a handle assembly including a first shaft having a distal portion and a locking recess formed at the distal portion; and
      a seal assembly including a shield member, wherein the seal assembly is movable relative to the handle assembly between (a) a first position in which the shield member radially shields the locking recess and (b) a second position in which the shield member is axially spaced apart from the locking recess.
18. The syringe of example 17 wherein the shield member further comprises a ramped unlocking projection, wherein the first shaft further comprises a locking projection, and wherein the locking projection is configured to mate with the unlocking projection in the first position.
19. The syringe of example 17 or example 18, further comprising a locking assembly including a lock plate configured to engage the locking recess when the seal assembly is in the second position to lock the position of the plunger assembly within the barrel.
20. The syringe of example 19 wherein the shield member includes a ramped unlocking feature, and wherein movement of the seal assembly from the second position to the first position is configured to move the ramped unlocking feature relative to the locking recess to drive the lock plate from the locking recess to permit movement of the plunger assembly through the barrel.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1-16. (canceled)

17. A syringe, comprising:
a barrel; and
a plunger assembly slidably positioned within the barrel, wherein the plunger assembly comprises—
a handle assembly including a shaft having a distal portion and a locking recess formed at the distal portion; and
a seal assembly including a shield member, wherein the seal assembly is movable relative to the handle assembly between (a) a first position in which the shield member radially shields the locking recess and (b) a second position in which the shield member is axially spaced apart from the locking recess.

18. The syringe of claim 17 wherein the shield member further comprises a ramped unlocking projection, wherein the shaft further comprises a locking projection, and wherein the locking projection is configured to mate with the unlocking projection in the first position.

19. The syringe of claim 17, further comprising a locking assembly including a lock plate configured to engage the locking recess when the seal assembly is in the second position to lock the position of the plunger assembly within the barrel.

20. The syringe of claim 19 wherein the shield member includes a ramped unlocking feature, and wherein movement of the seal assembly from the second position to the first position is configured to move the ramped unlocking feature relative to the locking recess to drive the lock plate from the locking recess to permit movement of the plunger assembly through the barrel.

21. The syringe of claim 17 wherein the shaft is a first shaft, wherein the plunger assembly further comprises a second shaft coupled to the seal assembly, and wherein the first shaft is movable relative to the second shaft.

22. The syringe of claim 21 wherein the plunger assembly further comprises a biasing member operably coupling the first shaft to the second shaft.

23. The syringe of claim 22 wherein, in the second position, the biasing member is compressed to store potential energy.

24. The syringe of claim 22 wherein the biasing member biases the seal assembly to the first position.

25. A plunger assembly, comprising:
a handle assembly including a first shaft having a distal portion;
a seal assembly including a second shaft and a seal head coupled to the second shaft; and
a biasing member operably coupling the first shaft to the second shaft, wherein the biasing member has a biasing force, and wherein—
the biasing force is configured to bias the seal assembly to a first position that is axially toward the distal portion of the first shaft; and
a force on the seal assembly greater than the biasing force is configured to move the seal head to a second position that is axially away from the distal portion of the first shaft.

26. The plunger assembly of claim 25 wherein the biasing member is a compression spring.

27. The plunger assembly of claim 25 wherein, in the first position, the seal head engages the distal portion of the first shaft.

28. The plunger assembly of claim 25 wherein, in the second position, the seal head and the distal portion of the first shaft define a locking recess therebetween.

29. The plunger assembly of claim 25 wherein the second shaft extends at least partially through the first shaft.

30. The plunger assembly of claim 25 wherein the second shaft comprises a visual indicator, wherein the first shaft includes an aperture, and wherein the visual indicator is at least partially visible through the aperture when the seal head is in the second position.

31. A clot treatment system, comprising:
an aspiration catheter defining a lumen;
a tubing assembly fluidly coupled to the lumen of the catheter, wherein the tubing assembly includes a valve that is movable between (a) a first position permitting fluid flow through the tubing assembly from the lumen and (b) a second position inhibiting fluid flow through the tubing assembly from the lumen; and
a syringe including a barrel, a plunger assembly slidably positioned within the barrel and movable between a first position and a second position, and a locking assembly coupled to the barrel, wherein—
the barrel is configured to be fluidly coupled to the tubing assembly,
with the valve in the first position,
the plunger assembly is configured to be withdrawn through the barrel to generate vacuum pressure in the barrel,
the plunger assembly is configured to move from the first position to the second position in response to the vacuum pressure, and
the plunger assembly is configured to lock to the locking assembly in the second position such that the locking assembly inhibits movement of the plunger assembly through the barrel, and
upon subsequent movement of the valve from the first position to the second position,
the plunger assembly is configured to move from the second position to the first position in response to the vacuum pressure being released, and
the plunger assembly is configured to unlock from the locking assembly in the first position such that the locking assembly permits movement of the plunger assembly through the barrel.

32. The clot treatment system of claim 31 wherein the plunger assembly includes a sealing head, a shaft, and a biasing member operably coupling the sealing head to the shaft, and wherein in the first position the biasing member biases the sealing head into engagement within the shaft.

33. The clot treatment system of claim 31 wherein the plunger assembly includes a sealing head, a shaft, and a biasing member operably coupling the sealing head to the shaft, and wherein in the second position the vacuum pressure moves the sealing head away from the shaft against a biasing force of the biasing member to define a locking recess between the shaft and the sealing head.

34. The clot treatment system of claim 33 wherein the locking assembly includes a lock plate configured to be positioned in the locking recess when the plunger assembly is in the second position such that the lock plate inhibits movement of the plunger assembly through the barrel.

35. The clot treatment system of claim 35 wherein movement of the plunger assembly from the second position to the first position is configured to drive the lock plate from the locking recess to permit movement of the plunger assembly through the barrel.

36. The clot treatment system of claim 31 wherein the valve comprises a user-actuatable stopcock.
